(19) United States
(12) Patent Application Publication
(10) Pub. No.: US 2018/0353505 A1
(43) Pub. Date: Dec. 13, 2018

(54) COMPATIBLE SOLUTE OR SOLUTE MIXTURE FOR USE IN THE PREVENTION OR TREATMENT OF DISEASES HAVING BARRIER DEFECTS IN EPITHELIAL TISSUES

(71) Applicant: bitop AG, Witten (DE)

(72) Inventors: Andreas Bilstein, Bergheim (DE); Olaf Scherner, Essen (DE)

(73) Assignee: bitop AG, Witten (DE)

(21) Appl. No.: 15/780,964

(22) PCT Filed: Dec. 2, 2016
(86) PCT No.: PCT/EP2016/079548

§ 371 Date: Jun. 1, 2018

(30) Foreign Application Priority Data
Dec. 3, 2015 (DE) 10 2015 121 050.9

(51) Int. Cl. A61K 31/505; A61K 8/49; A61Q 17/00; A61P 37/08; A61P 39/00; A61Q 19/00

(52) U.S. Cl. CPC A61K 31/505; A61K 8/4953; A61Q 19/007; A61P 37/08; A61P 39/00; A61Q 17/00

(57) ABSTRACT

The invention relates to a compatible solute or a solute mixture as well as to a composition comprising at least one solute or one solute mixture for use in the prevention or treatment of barrier malfunctions of epithelial tissues associated with at least one biobased noxa, in particular of diseases comprising at least one barrier malfunction in at least one cell layer of at least one epithelial tissue, wherein at least one solute is selected from compounds of formula I, of formula II, physiologically compatible salts of formula I, formula II, stereoisomeric forms of the compounds of formula I, formula II, and physiologically compatible salts of the stereoisomeric forms, or a mixture of at least two of the afore-mentioned compounds. The at least one compatible solute, solute mixture and/or the compositions are provided in the form of a cosmetic, medical product, medicament, of an additive to one of the afore-mentioned products or as component of an in-vitro diagnostic product (IVD).

The invention relates to a compatible solute or solute mixture as well as to a composition comprising at least one solute or one solute mixture for use in the prevention or treatment of barrier malfunctions of epithelial tissues associated with at least one biobased noxa, in particular of diseases comprising at least one barrier malfunction in at least one cell layer of at least one epithelial tissue, wherein at least one solute is selected from compounds of formula I, of formula II, physiologically compatible salts of formula I and formula II, stereoisomeric forms of the compounds of formula I, formula II, and physiologically compatible salts of the stereoisomeric forms, or a mixture of at least two of the afore-mentioned compounds. The at least one compatible solute, solute mixture and/or the compositions are provided in the form of a cosmetic, medical product, medicament, of an additive to one of the afore-mentioned products or as component of an in-vitro diagnostic product (IVD).

Compatible solutes, also referred to as osmolytes, are organic compounds having low molar mass. They are used as protective substances in cosmetic compositions as well as in medical products and medicaments.

Human are exposed to constant influence form the environment. Influences from the environment act on humans as living or non-living factors. Living factors are e.g. insects, parasites and pests, pets and plants which may cause health impairment of human by influence on human organism. Non-living factors are components or compounds from the environment or from nature, respectively. Usually, non-living factors are e.g. excretions of living factors, such as faeces, toxins or components of living factors, such as pollen, needles, secretions etc. If these non-living factors come into contact with the human organism, they may also cause health impairment of human. All in all, the afore-mentioned living and non-living factors may be summarised as biobased noxae.

Exposure of human and animal organism by biobased noxae is omnipresent. Environment, space, air and water are enriched with a variety of different biobased noxae on the way to work, at work, during leisure time and on vacation. The use of ventilating systems directing the outside air into the interior or by air conditioning systems support circulation and distribution of biobased noxae. The exposure with biobased noxae also varies due to seasonal differences depending on the season, temperature, wind and/or humidity.

Biobased noxae may cause different diseases having typical symptoms. Frequently, the reactions appear on, under and/or in the skin. A reaction of the skin or generally skin alteration is also referred to as efflorescence. Efflorescences are primarily visible and/or palpable basic elements of a disease of the skin and/or of the subcutis, and comprise primary efflorescences (or primary efflorescences), i.e. efflorescences that has developed from healthy skin without intermediate stage, and secondary efflorescences (or secondary efflorescences), thus efflorescences that has developed from primary efflorescences.

If the biobased noxae were taken up, in particular via the skin, mucosa and/or systemically, the resulting disease appears in symptoms, such as e.g. nausea, diarrhea, fever, oedemata, rednesses etc. The diseases comprise superficial infections, inner inflammations, pulmonary inflammations, Asthma, diseases of the bronchial tubes, diseases of the subcutis, diseases of the gastro-intestinal mucosa etc.

The diseases caused by biobased noxae have in common that the respective epithelial tissue is damaged at least in part. The most common cause is am impaired, defective or destroyed barrier function (selective permeability barrier) of the affected epithelial tissue. Epithelium is the tissue coating the inner and outer surface of the body and accomplishing the function of a barrier for foreign substances, such as e.g. biobased noxae. Therefore, epithelial cells or epithelial tissues, respectively, functions as barrier against mechanical injuries, penetrating noxae, fluid loss and evaporation.

Characteristic of individual epithelial cells and epithelial tissues is their polarity. The epithelium (synonymously epithelial tissue) consists of polar cells having a basal (synonymously basolateral) side an apical (lumen) side. On the basal side, they are in contact with the basal membrane and, laterally, with other cells via cell contacts. Epithelial tissues do not have blood vessels, but the detection of cytokeratins is characteristic of the different epithelial tissues.

The barrier provided by epithelial cells, in particular selective permeability barrier, and cohesion are strengthened by intercellular junctions. A distinction is made between four groups of cell junctions being involved in formation of the barrier in the epithelial tissue, in particular selective permeability barrier: tight junctions (Zonula occludens), adherens junctions or belt desmosomes (Zonula adhaerens), desmosomes and gap junction. The so-called epithelial junctional complex is formed by a unit (complex) of Zonula occludens, Zonula adhaerens and desmosomes and occurs in most of the single-layered epithelia. Due to the epithelial junctional complex, a selective permeability barrier is ensured in the space between the somatic cell (synonymously intercellular space) preventing an uncontrolled paracellular substance transport between the somatic cells. Penetration by biobased noxae is thereby controlled or prevented, respectively.

If the barrier described afore, in particular selective permeability barrier, has gaps, or if the penetrating noxae are such small that they nevertheless pass the barrier, health impairments arise therefrom within the meaning of the invention.

The affected person has different medicaments, medical products and cosmetics at its disposal for treatment of such health impairments. These preparations often contain steroids, antibiotics, antimycotics, analgesics and/or further synthetic agents and excipients. The afore-mentioned preparations treat the infection e.g. by attacks on parasites, bacteria, viruses or fungi. However, recovery of the barriers of the affected epithelial tissues is not supported by these agents. Endogenous mechanism, the immune system or specific expression factors have to recover the barrier instead. Furthermore, the aforementioned classes of substance have the disadvantage of frequently having severe side effects and/or causing allergic reactions in human.

Thus, regeneration of the epithelial, selective permeability barrier described afore relies on endogenic vitality for recovery of the barrier function of epithelial tissues. But after a disease progression and due to potentially occurring side effects, the endogenic vitality usually is reduced and the body may only very slowly or is not able at all to maintain or recover the barrier of epithelial tissues.

Up to now, no detailed mechanism of actions has been described having a positive impact on the quality of the epithelial barrier (in particular selective permeability barrier or barrier function in epithelial tissues) in the case of health impairments due to the influence of biobased noxae. Nor for the afore-mentioned class of substance.

It is also the object of the present invention to provide a compound or a mixture for use in the prevention or treatment of the afore-mentioned health impairments and diseases comprising at least one barrier malfunction in at least one epithelial tissue. Moreover, a composition containing the compound or the mixture described afore for use in the prevention or treatment of efflorescences of human or animal skin and mucosa shall be provided. Another object of the present invention is to provide cosmetic formulations and formulations for medical products and medicaments containing the solute described afore and/or the compositions described afore comprising the at least one compound for oral, nasal or topic administration. It is the object of the invention to provide cosmetic products, medical products and medicaments for prevention or treatment of barrier malfunctions of epithelial tissues associated with at least one biobased noxa, in particular of diseases comprising at least one barrier malfunction in at least one cell layer of at least one epithelial tissue. It is therefore the object of the present invention to provide a compound or a mixture that is suitable in minimizing, preventing or treating malfunctions of the barrier function in epithelial tissues. It is also the object of the present invention to provide a compound or a mixture that protects epithelial tissues against negative influences by biobased noxae. In this context, the compound or the mixture shall stabilise the barrier such that uncontrolled penetration of biobased noxae is inhibited or prevented. A compound or a mixture for use in the prevention or treatment of interferences of the barrier function caused by biobased noxae or associated with at least one biobased noxa shall be provided. It is an object of the present invention to protect and/or stabilise stability, regeneration and function of the epithelial barrier in outer epithelial tissues comprising outer skin, in transitional epithelial tissues comprising oral and nasal cavity and/or in inner epithelial tissues comprising lower respiratory tract and inner endothelium. Furthermore, a compound or a mixture preventing or reducing efflorescences caused by biobased noxae, neurodermatitis, inflammations of the skin, of the eye, of the oral/nasal mucosa, respiratory diseases, fluid loss, dehydration of the mucosa or skin, conjunctiva, cornea and/or allergic reactions of epithelial tissues shall be provided.

Surprisingly, it has now been found that the compatible solute ectoine and its derivatives have such effectiveness.

Therefore, one subject matter of the present invention is a compatible solute or solute mixture for use in prevention or treatment of barrier malfunctions of epithelial tissues associated with at least one biobased noxa, preferably impaired and reduced selective permeability barrier, in particular of diseases comprising at least one barrier malfunction in at least one cell layer of at least one epithelial tissue, comprising at least one compound selected from compounds of formula I, of formula II, physiologically compatible salts of formula I and formula II, stereoisomeric forms of the compounds of formula I, formula II and physiologically compatible salts of the stereoisomeric forms, or from a mixture of at least on two of the afore-mentioned compounds, wherein in formula I

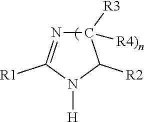

and in formula II

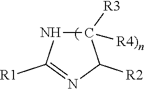

R1=H or alkyl,

R2=H, COOH, COO-alkyl or CO—NH—R5,

R3 and R4 each independently H or OH,

R5=H, alkyl, an amino acid residue, dipeptide residue or tripeptide residue

n=1, 2 or 3, and alkyl=an alkyl group having C₁-C₄ carbon atoms.

Within the meaning of the invention, alkyl comprises linear, cyclic and branched alkyl groups comprising methyl (—CH3), ethyl (—C2H5), propyl (—CH2CH2CH3 or —CH(CH3)₂), and butyl (—CH2CH2CH2CH3, H3C(CH)CH2CH3, —CH2CH(CH3)₂ and C(CH3)₃). Linear alkyl groups are preferred, and the methyl group is particularly preferred.

Amino acid residues derive from the appropriate amino acids and their stereoisomeric forms, such as L- and D-forms, and comprise the amino acids alanine, ss-alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine, y-aminobutyrate, Ne-acetyllysine, NS-acetylornithine, Ny-acetyldiaminobutyrate, and Na-acetyldiaminobutyrate. L-amino acids, such as L-cysteine, L-valine, L-arginine, L-asparagine, L-histidine, L-tryptophane, L-phenylalanine, and L-lysine are preferred. The amino acid residues of amino acids alanine, L-alanine, asparagine, aspartic acid, glutamine, glutamic acid, glycine, serine, threonine, valine, y-aminobutyrate, N-Acetyllysine, N-acetylornithine, Ny-acetyldiaminobutyrate, and Na-Acetyldiaminobutyrate are particularly preferred.

Dipeptide residues are composed of two amino acids and comprise linear and cyclic dipeptide residues, wherein linear dipeptide residues have one and cyclic dipeptide residues have two peptide bond(s). Tripeptide residues are composed of three amino acids and comprise linear and cyclic tripeptide residues, which have three peptide bonds in the case of linear structure or four peptide bonds in the case of cyclic structure. A peptide bond is an amide bond (—CO—NH—) between the nitrogen atom of the amine group of a first amino acid and an oxygen atom of the carboxy group of a second amino acid. Preferred dipeptide residues and tripeptide residues are composed of the afore-mentioned amino acids and, particularly preferably, of the particularly preferred amino acids described before.

Physiological salts of the compound of formula I and of the compound of formula II comprise alkali, earth alkali or ammonium salts, such as Na-, K-, Mg- or Ca-salts, as well as salts derived from organic bases, e.g. aliphatic or aromatic amines, such as triethylamine or tris-(2-hydroxyethyl)-amine. Preferred physiologically compatible salts of the compounds of formula I and of formula II are obtained by reaction with inorganic acids, such as hydrochloric acid, sulfuric acid and phosphoric acid, or with organic carboxylic acids or sulfonic acids, such as acetic acid, citric acid, benzoic acid, maleic acid, fumaric acid, tartaric acid, and p-toluene sulfonic acid.

Another subject matter of the present invention is the use of the compounds of formula I and of formula II, physiologically compatible salts of formula I and formula II, stereoisomeric forms of the compounds of formula I, formula II, and physiologically compatible salts of the stereoisomeric forms, or of a mixture, as well as of preferred embodiments described herein, for the production of a cosmetic product, medical product, in-vitro diagnostic products (IVD), medicaments and/or of an additive or component to one of the mentioned products for prevention or treatment of barrier malfunctions of epithelial tissue associated with at least one biobased noxa, in particular of diseases comprising at least one barrier malfunction in at least one cell layer of at least one epithelial tissue. Preferred diseases with an altered selective permeability barrier comprise a reduced Claudin-expression.

Compounds wherein R1 is a hydrogen atom or a methyl group (CH3), R2 is a hydrogen atom or COOH, R3 and R4 are, each independently, a hydrogen atom or OH, and n is equal to 2 are preferred compounds of formula I and of formula II. In particular, preferred compounds according the afore-mentioned definitions include 1,4,5,6-tetrahydro-2-methyl-4-pyrimidine carboxylic acid (ectoine) und 1,4,5,6-tetrahydro-5-hydroxy-2-methyl-4-pyrimidine carboxylic acid (hydroxyectoine), as well as physiologically compatible salts and stereoisomeric forms of the afore-mentioned compounds. The isomeric compounds of formula I and of formula II, (S)-1,4,5,6-tetrahydro-2-methyl-4-pyrimidine carboxylic acid (S-ectoine) und (S,S)-1,4,5,6-tetrahydro-5-hydroxy-2-methyl-4-pyrimidine carboxylic acid ((S,S)-hydroxyectoine), are preferred.

Compounds of formula I and of formula II, wherein R1 is a hydrogen atom or a methyl group (CH3), R2 is a hydrogen atom or COOH, R3 and R4 are, each independently, a hydrogen atom or OH, and n is equal to 3 or 4, are more preferred for use in the prevention or treatment of barrier malfunction of epithelial tissues caused by biobased noxa, in particular diseases comprising at least one barrier malfunction in at least one epithelial tissue. In particular, preferred compounds according to the afore-mentioned definitions include (S)-4,5,6,7-tetrahydro-2-methyl-1H-[1,3]-diazepine 4-carboxylic acid (homoectoine) with n equal to 3 und 3,4,5,6,7,8-hexahydro-2-methyl-1,3-diazocine 4-carboxylic acid (HHMDCA) with n equal to 4, as well as physiologically compatible salts and stereoisomeric forms of the afore-mentioned compounds.

The compounds described before can be present as optical isomers, diastereomers, racemates, zwitterions, cations, or as a mixture of at least two of the afore-mentioned forms. Isomers comprise (R,R)-, (R,S)-, (S,S)- and (S,R)-configurations of afore-mentioned compounds. Aside from isomers, diastereomers, racemates, zwitterions, cations, and mixtures of the afore-mentioned compounds are also a subject matter of the invention. Derivatisations can be performed with hydroxy acid derivatives, sulfonic acid derivatives, carboxylic acid derivatives, such as amides, esters etc., carbonyl-, ether-, alkoxy- and hydroxyl-groups. A possible derivative, without being limited thereto, is (S,S)-alpha-amino-beta-hydroxyectoine.

The terminology “solute” shall be synonymously understood as “compatible solute”, and “mixture” shall be synonymously understood as “solute mixture”. “Solute mixture” is always related to a mixture of at least two solutes of formula I and/or formula II. “Ectoine”, “hydroxyectoine” and “homoectoine” always comprise all stereoisomeric forms of the respective compound. Specific isomers are marked as such.

In an embodiment according to the invention of the use of the compatible solute or solute mixture, the at least one biobased noxa is selected from animal noxae, plant noxae, noxae of insects and pests, microbial noxae, environmental noxae, food noxae, components and/or compounds of the afore-mentioned noxae respectively and/or combinations of at least two of the biobased noxae.

Within in the meaning of the invention, biobased noxae are exclusively of natural origin, originating from nature and of biogenic origin. This also includes temperatures and humidity amounts induced by wind and weather in the environment. Within the meaning of the invention, biobased noxa preferably are biobased noxae which make contact with the human or animal organismal at least via the outer epithelial tissue. Preferably, the biobased noxae are peptidebased. If biobased noxae exclusively act via the outer contact with the outer epithelial tissue, they are contact noxae. Other noxae act also or just after intake via the outer epithelial tissue and/or via the mucosa (mouth, nose, eye, gastro-intestine). Biobased living noxa (plants, insects, pests, bacteria, viruses, fungi, yeasts, each reproducible) may be distinguished from biobased non-living noxae. The last-mentioned comprise animal, microbial and plant excretions including toxins, compounds, faeces, secretions, excreta, food components and physical noxae such as wind, temperature, humidity and sun (photodynamic exposure to light). Biobased noxae also comprise biobased haptens which may only act in combination with a protein as noxa in human organism. The protein may be another biobased noxa according to the invention or an endogenous protein of the affected human. Preferably, biobased noxae, in particular biobased contact noxae, are peptidebased.

Preferably, the at least one noxa or one combination of at least two biobased noxae is selected from at least one of the groups
a) animal noxae comprising domestic animals, cat, dog, pig, rodents, farm animals, pig, goat, excretions of the afore-mentioned animals, animal epithelia and/or animal hair,
b) plant noxae comprising flower pollen, fruit organs, saps, secretions, poisons, resins, odorous substances, flavouring substances, toxins, stinging hairs, hooks, needles, spines, components and/or compounds of plant noxae,
c) noxae of insects and pests comprising mites, dust mites, excretions of insects, pests and/or parasites, bee glues, bee venoms, wasp venoms, spider venoms, pest bites, mosquito stings, horsefly stings and ant stings or bites respectively, components, such as e.g. spines, poisonous hairs, barbs, and/or compounds of insects or pests,
d) microbial noxae comprising microorganisms, fungi, yeasts, Malassezia species, bacteria, Staphylococcus aureus, mould spores, moulds, bacterial toxins, delta toxin, antibiotics, viruses, mycotoxins, components and/or compounds of microorganisms,
f) environmental noxae such as photodynamic exposure to light, wind, seasonal temperature fluctuation,
e) food noxae comprising nuts, peanuts, hazelnuts, wine, cow's milk, wheat, soy, hen's egg, protein, fish, shellfish, crustaceans, molluscs, raw vegetables, raw fruits, components and/or compounds of food, and/or
f) in particular human noxae comprising endogenous components/compounds, sweat, proteins and/or amino acids.

The respective noxae themselves may have a negative impact on the barrier function of epithelial tissues (Table 2), in particular on the selective permeability barrier, or may only have a negative to damaging impact on epithelial tissues by a combination of at least two of the afore-mentioned noxae. In particular, secondary, partially stronger, barrier malfunctions (e.g.infections caused by infectious noxae e.g. of microorganisms and allergies caused by non-infectious noxae) may follow on primary damages of the barrier function (e.g. mechanical lesions by environmental noxae, such as high temperature, strong cold and/or wind).

TABLE 1
Biobased noxae within the meaning of the invention

| Group | Individual noxae |
|---|---|
| a) | domestic animals, such as cat, dog, pig, rodents, etc.; farm animals, pig, goat, |
| animal | goose etc.; excretions of the afore-mentioned animals (faeces, urine, sweat, |
| noxae | body fluids), animal epithelia, animal hair (adnexa, skin scales) |
| | marine animals: jellyfishes, sea urchins, stingrays, cone snails and weavers; |
| | sponges (silicic acid needles), sea anemones, cnidarians, such as jellyfishes, |
| | scyphozoans, moon jellyfish and box jellyfish as well as sea wasps and their |
| | poisons (contact to tentacles) respectively, cnidocytes, spines, lion's mane |
| | jellyfish, mauve stinger and compass jellyfish; algae and their excretions and |
| | toxins |
| b) | flower pollen, fruit organs, saps, secretions, poisons, resins, natural rubber, |
| plant | latex, odorous and flavouring substances, toxins, stinging nettle and other |
| noxae | irritating plants (e.g. arnica, mugwort, chamomile and yarrow), resins, stinging |
| | hairs, hooks, needles, spines, components and/or compounds of plant noxae, |
| | herbal substances, e.g. Peru balsam, (Balsamum peruvianum), tree moss |
| | (Evernia furfuracea), eucalyptus etc.; wool wax alcohol, lanolin, arnica, |
| | chamomile, bee glue, marigold |
| | grass pollen, birch pollen, hazel pollen are common triggers for allergies against: |
| | stone fruits (apple, pear), pip fruits (plum, cherry, peach), peanut/hazelnut, brazil |
| | nut, walnut, almond, celery, carrot, kiwi, spices, such as anise, curry; grass |
| | pollen are common triggers for allergies against: wheat flour, peanut, soy (bean, |
| | flour, milk); |
| | herb pollen are common triggers for allergies against: celery, carrot, fennel, |
| | garlic; chamomile, parsley; sunflower seeds; spices, such as caraway, curry, |
| | paprika, anise, pepper, nutmeg, cinnamon, ginger, coriander |
| c) | mites, dust mites and mite excretions; ant, spider, worms, lices, etc. pest bites, |
| insects, | pest liquids, ant venoms, spider venoms, bee/wasp venoms (allergens: |
| pests | glycoproteins, phospholipase A1 and A2, hyaluronidase, melittin, apamin, MCD |
| | peptide 401, antigen 5), spider stings, mosquito stings and ant stings and/or |
| | bites respectively, bee glue; pest and insect components, such as e.g. spine, |
| | poisonous hairs, barbs |
| d) | microorganisms, fungi, yeasts, infectious microorganisms, Malassezia species, |
| microbial | bacteria, Staphylococcus aureus, mould spores, moulds, bacterial toxins, delta |
| noxae | toxin, antibiotics, viruses, mycotoxins, components and/or compounds of |
| | microorganisms, exotoxins, endotoxins, Corynebacterium diphtheriae |
| e) | nuts, peanuts, hazelnuts, wine, cow's milk, wheat, soy, hen's egg, protein, fish, |
| food | shellfish, crustaceans, mollusks, raw vegetables, raw fruits, components and/or |
| noxae | compounds of food |

In further embodiment of the use according to the invention of the at least one compatible solute or solute mixture, the at least one biobased noxa is peptidebased. Within the meaning of the invention peptidebased means that the biobased noxa is a compound or comprises at least one compound which comprises at least one peptide bond between at least two amino acids.

In a further embodiment of the use according to the invention of the at least one compatible solute or solute mixture, epithelial tissues, in particular surface epithelia as being the boundary between inner and outer surfaces as well as between functional unities,
outer epithelial tissues comprising skin, outer skin, scalp, epidermis, nail bed, nail body (eponychium), cornea and conjunctiva, and in particular mucosa, of the eye, outer ear, external auditory canal, and lips,
transitional epithelial tissue comprising oral cavity, oral mucosa, gingiva, tongue, tongue mucosa, upper respiratory tract, nasal cavity, paranasal sinuses, nasal mucosa, voice flaps, throat and genitals, and/or
inner epithelial tissue comprising lower respiratory tract, trachea, bronchial tubes, bronchial tree, lungs, inner endothelial tissue, continuous endothelium, in particular continuous endothelium of lung and heart, endothelium of heart blood vessels, heart lymph vessels, oesophagus, gastric mucosa, and/or small/intestinal mucosa.

Epithelial tissues are histologically divided into a single-layered, multi-layered and multi-row epithelium by the number of cell layers. Furthermore, the epithelium is divided into a flat epithelium, isoprismatic or cubic epithelium and highprismatic or cylindrical epithelium, respectively, by the form of the cells. The degree of keratinisation is described as keratinized or unkeratinized. The epithelium has a characteristic histology depending on localisation and function of the epithelium. The occurrence of the epithelial tissues comprised within the meaning of the invention is summarized below, such that when the term epithelial tissue is used within the meaning of the invention, the following summary is decisive, unless it is explicitly referred to a particular embodiment.

TABLE 2
Epithelial tissues within the meaning of the invention

| Epithelial tissue | Occurance |
|---|---|
| Single-layered | Serous membranes comprising pleura, visceral pleura, pericardium, tunica |
| squamous | vaginalis of the testis; alveolar epithelium, endothelium, lung (endothelium), |
| epithelium | endothelium of heart, blood and lymph vessels; tongue mucosa (ventral |
| | surface of the tongue) |
| Single-layered | Epithelium of stomach, small and large intestine; gastric mucosa, intestinal |
| high-prismatic | mucosa |
| epithelium | |
| Two-row | Salivary gland, oral cavity, lacrimal duct |
| epithelium | |
| Multi-row high- | Nasal cavity, gullet (throat), larynx, respiratory passages, bronchial tree, |
| prismatic | auditory tube, urethra, |
| epithelium | |
| Multi-layered un- | anterior corneal epithelium (eye), vocal fold, oral cavity, gingiva (inner |
| keratinized | junctional epithelium surrounding the dental neck), throat, oesophagus, |
| epithelium | anus, vagina, tongue mucosa (dorsal surface of the tongue); conjunctiva |
| | (conjunctiva) of the eye, cornea |
| Multi-layered | epidermis (Stratum basale, Stratum spinosum, Stratum granulosum), nasal |
| keratinized | vestibule, external auditory canal, gingiva (outer junctional epithelium to the |
| epithelium | oral cavity), |

The epithelial tissues of Table 2 comprise tight junction, adherens junctions and/or desmosomes as intercellular junctions. The tight junctions thereby achieve the actual paracellular barrier function of the epithelial tissues, in particular selective permeability barrier, whereby transcytosis is enabled and loss of body fluids is prevented at the same time. Furthermore, paracellular penetration of molecules, ions, antigens, peptidebased noxae and microorganisms is prevented. The selective resorption and secretion of nutrients, electrolytes and water is an important function. The cells are connected and thus mechanically stabilised by adherens junctions and desmosomes. Tight junction also contributes mechanical stabilization of the epithelial tissue besides their function in the selective permeability barrier. Therefore, the focus of the present invention is on analysis of permeability properties and impact on the barrier function of epithelial tissues.

Therefore, a further subject matter of the present invention is a compatible solute or solute mixture for use according to the invention, wherein the at least one barrier malfunction of at least one epithelial tissue has an impaired intercellular cell structure in at least one cell layer of the epithelial cells of the outer epithelial tissue, transitional epithelial tissue and/or inner epithelial tissue. Preferably, the at least one epithelial tissue comprises tight junction, adherens junction and/or desmosomes as intercellular junctions. Malfunctions of the afore-mentioned junctions result in structural weakening and thus in an impaired and reduced selective permeability barrier.

Due to their versatile functions, tight junctions are the most important components in maintenance of the barrier function of epithelial tissues. They circularly surround epithelial cells at the apical end as intramembranous, continuous structure. Considering the spatial arrangement of intercellular junctions in epithelial tissues, tight junction is most apically arranged in all epithelial tissues, followed by adherens junction, desmosomes and gab junction as being the basally arranged intercellular junction. Thus, biobased noxa primarily act on tight junction. Only after the tight junction are damaged or defective and the biobased noxa may penetrate due to a missing permeability barrier, the biobased noxa acts on the adherens junction etc.. If the afore-mentioned epithelial tissues or epithelial cells, respectively, have malfunctions in the barrier (synonymously selective permeability barrier), damage or loss of tight junction is primarily to be assumed (see Examples 1 to 3).

Therefore, a further subject matter of the present invention is a compatible solute or solute mixture for use according to the invention, as described, wherein the at least one barrier malfunction of the at least one epithelial tissue is a damage of the tight junction in at least one cell layer. In particular, damage of the tight junction results in an impaired and reduced selective permeability barrier, in particular in the intercellular spaces.

In a further embodiment of the present invention using the at least one compatible solute or solute mixture, the at least one barrier malfunction of at least one epithelial tissue has an impaired intercellular cell structure, in particular an increased permeability, in at least one cell layer of the epithelial cells of the outer epithelial tissue, transitional epithelial tissue and/or inner epithelial tissue. Preferably, the at least one barrier malfunction comprises a damage of the tight junction in at least one cell layer of the at least one epithelial tissue. Damages of the tight junction may be a low protein stability, modulation of proteins, in particular of the occludin and/or claudin protein family, partial degradation or breaks of the transcellular loop of the tight junction proteins, whereby the cell structure is interfered and more permeable. The interference of tight junction proteins results in impairment and decrease of the selective permeability barrier of epithelial tissues, whereby an increased paracellular flow of biobased noxae results in tur (Example 2).

Therefore, in a further embodiment of the present invention using the at least one compatible solute or solute mixture, the at least one barrier malfunction of the at least one epithelial tissue is an impaired and reduced selective permeability barrier. An impaired or damaged selective permeability barrier, respectively, may be identified by a reduced transepithelial electric resistance (TEER), as shown in Example 1, and may be shown by lower expression of at least one protein of the claudin protein family, as shown in Example 2.

The at least one biobased noxa (Table 1) may be the cause for impairment of the barrier function and/or may result in stronger barrier malfunction in epithelial tissues and in diseases resulting thereof in the case of already predisposed epithelial tissues having weakened barrier function, e.g. in the case of outer epithelial tissues, such as epidermis, cornea and/or conjunctiva. Preferably, claudin-associated diseases with an altered selective permeability barrier comprising reduced claudin expression. Claudin-associated diseases comprise inflammatory intestinal diseases, kidney diseases, bacterial or viral infections as well as skin diseases. The epithelial tissues have a reduced selective permeability barrier, among other things based on down-regulated claudin expression, in particular in the case of psoriasis, dermatitis and atopic dermatitis and related skin diseases with barrier impairment, in particular in the early stage.

As already explained above, regeneration of the epithelial, selective permeability barrier relies on endogenic vitality for recovery of the barrier function of epithelial tissues. But after a disease progression and due to potentially occurring side effects, the endogenic vitality usually is reduced and the body may only slowly or is not able at all to recover or maintain the barrier of epithelial tissues. The experiments described herein show that ectoine may support the body in regeneration of the selective permeability barrier being proved by increase of claudin expression and increased TEER (Examples 1 to 3).

In a further embodiment of the use according to the invention of at least one compatible solute or solute mixture, the at least one epithelial tissue having at least one barrier malfunction, preferably in at least one cell layer, has a reduced transepithelial electric resistance (TEER), which is measured in [Ohm], as explained in Example 1, in comparison with a non-damaged epithelial tissue. In particular, according to the invention, the TEER of the afore-mentioned epithelial tissues is increased and/or stabilised by influence of the at least one solute. In the case of an intact selective permeability barrier, free diffusion through cell interstices (paracellular way) is restricted by the selective paracellular permeability barrier, the density of which is tissue-specific and is typically described by the transepithelial electric resistance (TEER). Density of the selective paracellular permeability barrier is effected by the tight junction. Adherens junction as well as desmosomes mechanically connect the cells with each other.

In Example 1, epithelial cells of oral mucosa (TR146 cells=transitional epithelial tissue), porcine renal epithelial cells (LLC-PK1=inner epithelial tissue) as well as human keratinocytes (HaCaT cells=outer epithelial tissue) were analysed as representatives for all epithelial tissues according to the invention (Table 2). A positive impact of ectoine on stabilization of the transepithelial electric resistance (TEER) could be proved in all types of epithelial tissues (FIG. 3, FIG. 4, FIG. 5 and FIG. 6).

In a further embodiment of the use according to the invention of at least one compatible solute or solute mixture, the at least one epithelial tissue having at least one barrier malfunction, preferably in at least one cell layer, has an increased permeability for at least one biobased noxa, as shown in Example 2 by the allergy prevention assay (APA), in comparison with a non-damaged epithelial tissue. In particular, according to the invention, the permeability of the afore-mentioned epithelial tissues is reduced by influence of at least one solute. In Example 2, human epithelial cells of oral mucosa as well as of nasal mucosa (TR146/RPMI-2650 cells=transitional epithelial tissue), rat bronchial epithelial cell (RLE=inner epithelial tissues) as well as human keratinocytes and rabbit corneal epithelial cells (HaCaT/SIRC cells=outer epithelial tissue) were analysed as representatives for all epithelial tissues according to the invention (Table 2). A positive impact of ectoine on reduction of the permeability (APA) could be proved in all types of epithelial tissues (Table 4).

In a further embodiment of the use according to the invention of at least one compatible solute or solute mixture, the at least one epithelial tissue having at least one barrier malfunction, preferably in at least one cell layer, has a lower expression of at least one protein of the claudin protein family, as shown in Example 3, in comparison with a non-damaged epithelial tissue. In particular, according to the invention, the claudin expression of the afore-mentioned epithelial tissues is enhanced and/or stabilised by influence of at least one solute. In Example 3, human keratinocytes (HaCaT cells) were analysed as representatives for the outer epithelial tissue. A positive impact of ectoine on claudin-1 expression could be proved (Table 5) after heat and thus drying stress (environmental noxae according to the invention). Thus, ectoine is able to support formation of tight junction by stimulation of the expression of proteins of the claudin protein family. The barrier function, in particular the selective permeability barrier, is thereby stabilised or recovered in already damaged epithelial cells.

Within the meaning of the invention, non-damaged epithelial tissues correspond to the controls of the present Examples and are epithelial tissues not having been interfered by influence of biobased noxae.

The afore-mentioned effectiveness of ectoine for use in prevention or treatment of barrier malfunction in epithelial tissues, in particular on stabilisation and/or recovery of the selective permeability barrier (according to Examples 1, 2 and 3) is preferably achieved with greater than or equal to 10 mM to less than or equal to 1 M of the at least one compatible solute, preferably ectoine, hydroxyectoine and/or its derivatives, preferably greater than or equal to 10 mM to less than or equal to 750 mM, greater than or equal to 10 mM to less than or equal to 500 mM, greater than or equal to 25 mM to less than or equal to 500 mM, greater than or equal to 50 mM to less than or equal to 500 mM. In particular 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, +/- 5 mM deviation respectively.

Consequently, ectoine and its derivatives are suitable for use in both prevention and treatment of barrier malfunctions. In particular of diseases comprising at least one barrier malfunction in at least one epithelial tissue, which is associated with at least one, preferably peptidebased, biobased noxa (Table 1).

In a preferred embodiment of the present invention, epithelial tissue exclusively comprises outer epithelial tissues comprising skin, outer skin, scalp, epidermis, nail bed, nail body (eponychium), cornea and conjunctiva, and in particular mucosa, of the eye, outer ear, external auditory canal and lips, as well as transitional epithelial tissue comprising oral cavity, oral mucosa, gingiva, tongue, tongue mucosa, upper respiratory tract, nasal cavity, paranasal sinuses, nasal mucosa, voice flaps, throat and genitals. At least one compatible solute or solute mixture selected from compounds of formula I and/or of formula II, preferably ectoine and/or hydroxyectoine, is used for prevention or treatment of at least one barrier malfunction, in particular of the selective damaged permeability barrier, of the preferred epithelial tissues associated with at least one, preferably peptidebased, biobased noxa (Table 1).

In a preferred embodiment of the present invention, in particular of the afore-mentioned selection, preferred outer epithelial tissues and transitional epithelial tissues are those having tight junction respectively. Tight junction and desmosomes are strongly represented in particular in stratum granulosum and stratum spinosum and form the most important barrier for humans for biobased, in particular peptidebased, noxae. In this case, barrier malfunctions have a reduced transepithelial electric resistance (TEER), an increased permeability (APA) and/or a lower expression of at least one protein of the claudin protein family, each in comparison with non-damaged epithelial tissue having an intact barrier function. Thus, within the meaning of the invention, barrier malfunctions are present at the boundary between apical and basolateral membrane of epithelial cells.

In a further embodiment of the use according to the invention of the at least one compatible solute or solute mixture of formula I and/or of formula II, the at least one solute or solute mixture protects and/or stabilises the barrier function of the at least one epithelial tissue, in particular the selective permeability barrier, inhibits and/or reduces at least the impairment of the barrier function, in particular in at least one epithelial tissue, and/or at least partially restores the impairment of the barrier function (see above, Example 1-3).

In a further embodiment of the use according to the invention of the at least one compatible solute or solute mixture of formula I and/or formula II, the at least one solute or solute mixture at least shows a protective effect on the barrier function of epithelial cells of the outer epithelial tissues (preferably skin, epidermis), of the transitional epithelial tissues (preferably oral and nasal mucosa), and/or of the inner epithelial tissues (preferably pulmonary, bronchial and gastric epithelium) at a concentration of greater than or equal to 1 mM to less than or equal to 1 M, inhibits the impairment of the barrier function and/or at least partially restores the impairment of the barrier function of the afore-mentioned epithelial cells.

The used concentration of the at least one compatible solute, prefearbly ectoine, hydroxyectoine and/or its derivatives, amounts to greater than or equal to 1 mM to less than or equal to 1 M, greater than or equal to 5 mM, greater than or equal to 10 mM, greater than or equal to 15 mM, greater than or equal to 20 mM, greater than or equal to 25 mM, greater than or equal to 30 mM, greater than or equal to 35 mM, greater than or equal to 40 mM, greater than or equal to 45 mM, greater than or equal to 50 mM, greater than or equal to 55 mM, greater than or equal to 60 mM, greater than or equal to 65 mM, greater than or equal to 70 mM, greater than or equal to 75 mM, greater than or equal to 80 mM, greater than or equal to 85 mM, greater than or equal to 90 mM, greater than or equal to 95 mM, greater than or equal to 100 mM, greater than or equal to 110 mM, greater than or equal to 120 mM, greater than or equal to 130 mM, greater than or equal to 140 mM, greater than or equal to 150 mM, greater than or equal to 200 mM, greater than or equal to 250 mM, greater than or equal to 300 mM, greater than or equal to 350 mM, greater than or equal to 400 mM, greater than or equal to 450 mM, greater than or equal to 500 mM to respectively less than or equal to 1 M, less than or equal to 950 mM, less than or equal to 900 mM, less than or equal to 850 mM, less than or equal to 800 mM, less than or equal to 750 mM, less than or equal to 700 mM, less than or equal to 650 mM, less than or equal to 600 mM, less than or equal to 550 mM.

Particularly preferred concentrations ranges are greater than or equal to 10 mM to less than or equal to 1 M, greater than or equal to 10 mM to less than or equal to 750 mM, greater than or equal to 10 mM to less than or equal to 500 mM, greater than or equal to 25 mM to less than or equal to 500 mM, greater than or equal to 50 mM to less than or equal to 500 mM of respectively at least one compatible solute, preferably ectoine, hydroxyectoine and/or its derivatives. In particular 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, +/- 5mM deviation respectively.

Barrier malfunctions may be the cause but also the result of a disease (skin symptom, skin disease), inflammatory reaction due to infections and/or allergies, infection (superficial, internal) and/or an allergy. In this context, an impaired barrier function of the respectively interfered epithelial tissues shows different symptoms (synonymously phenotype or phenotypical, respectively). These symptoms may occur isolated, locally restricted, distributed at particular points or widespread.

In a further embodiment of the use according to the invention of the at least one compatible solute or solute mixture of formula I and/or of formula II, the at least one barrier malfunction of at least one epithelial tissue therefore phenotypically comprises itching, skin discolourations, calor, calor generation, fever, rednesses, dry skin (xeroderma), ring-shaped skin alterations, small blisters, blisters, pustules, papules, pimples, each with or without suppuration, abscesses, fistulas, rash, scabs, raisings, swellings, scratches, stings, sloughing, desquamation, wheals, angio-oedema, quincke-oedema, urticaria, plaques, ulcers, furuncles, carbuncles, eczemas and/or Baghdad boils.

In the case of skin contact with animal noxae, body reactions cause symptoms appearing by burning pain, itching and swelling of the affect skin region. In particular, this is to be observed in the case of allergies to pets as well as in the case of contact with marine animals. Particularly in the case of contact with cnidarians, severe pain, skin rash and blistering, but also severe symptoms of poisoning, such as vomiting, fever, disorientation, circulatory disorders and cardiovascular failure, occur depending on the extend of stinging. Swellings, rednesses, blistering and tissue death are usual symptoms in the case of contact with poisonous animals and cnidarians.

Contact with food noxae may result in wheals (urticaria), nettle rash, redness, itching, quincke-oedema, skin symptoms, such as neurodermatitis and, in the case of neurodermatitis, in neurodermatitis attacks, urticaria and quince-oedema, urticaria and, in the region of throat, nose and ears, in sneezing attacks and running nose.

In a further embodiment of the use according to the invention of the at least one compatible solute or solute mixture of formula I and/ or of formula II, the barrier malfunction of epithelial tissues of the manner described afore associated with at least one biobased, preferably peptidebased, noxa comprises parasitical, bacterial and/or viral diseases, mycoses, lesions, dryness, irritations, inflammations, hypersensitivities and/or allergic reactions, each of the outer epithelial tissues, transitional epithelial tissues and/or inner epithelial tissues. Preferably, the barrier malfunctions have a damage of the tight junction (see above) in at least one cell layer of the epithelial tissues. Allergic reactions comprise allergic reactions to the noxae based in the invention (Table 1), cross allergies and contact allergies, prefearbly of the outer epithelial tissues comprising skin, outer skin, scalp, epidermis, nail bed, nail body (eponychium), cornea and conjunctiva, and in particular mucosa, of the eye, outer ear, external auditory canal and lips, as well as transitional epithelial tissue comprising oral cavity, oral mucosa, gingiva, tongue, tongue mucosa, upper respiratory tract, nasal cavity, paranasal sinuses, nasal mucosa, voice flaps, throat and genitals.

At least one compatible solute or solute mixture selected from compounds of formula I and/or of formula II, prefearbly ectoine and/or hydroxyectoine, is used for prevention or treatment of the afore-mentioned barrier malfunctions, in particular diseases, associated with at least one, prefearbly peptidebased, biobased noxa (Table 1). The preferred concentration ranges described apply here for the at least one solute accordingly.

A contact allergy is a reaction to the contact with an allergen starting within 48-72 hours, in which the allergen, in particular the biobased noxa and biogenic working materials (e.g. latex, in agriculture, forestry and fishery), intrudes and/or penetrates the epithelial tissue, preferably the epidermis. Contact allergic reactions appear in the case of appropriately disposed individuals, based on genetic or non-genetic factors, by the symptoms already described afore.

Further preferred is a use according to the invention of a compatible solute or solute mixture of formula I and/or formula II in the case of barrier malfunction of preferably outer epithelial tissues and transitional epithelial tissues respectively having tight junction. In particular barrier malfunctions of the epidermis, enhancedly having tight junction and desmosomes in stratum granulosum and stratum spinosum, and forming the most important barrier for humans for biobased, in particular peptidebased, noxae. In this case, barrier malfunctions have a reduced transepithelial electric resistance (TEER), an increased permeability (APA) and/or a lower expression of at least one protein of the claudin protein family, each in comparison with non-damaged epithelial tissue having an intact barrier function. The preferred concentration ranges apply here for the at least one solute accordingly.

In a further embodiment of the use according to the invention of the at least one compatible solute or solute mixture of formula I and/or of formula II, at least one barrier malfunction is present with diseases, of respectively
outer epithelial tissues (Table 2) comprising diseases of the skin and the subcutis, skin lesions, infections of the skin and/or of the subcutis, mycoses, dry skin, in particular dry outer skin of the extremities/limbs, such as legs, feet, arms, hands, crooks of the arm, hollows of the knee, of the face, head and of the neck, contact allergies, dermatitis, eczemas, neurodermatitis, psoriasis, urticaria, herpes, orofacial herpes, diseases of the eye, inflammations of the conjunctiva and/or cornea, conjunctivitis, keratitis, dry conjunctiva, diseases of the outer ear, inflammations of the outer ear and/or of the external auditory canal, Otitis externa, physical impairments and/or damages of the skin structure comprising injuries, stings, slashes, scratches, abrasions, burns and/or chemical burns, each triggered by contact with at least one biobased, prefearbly peptidebased, noxa,
transitional epithelial tissues (Table 2) comprising allergic reaction of the nasal mucosa, allergic reactions of at least one mucosa of the oral cavity (oral, tongue and/or gingival epithelium), dry nasal mucosa, diseases of the oral cavity, cysts, phlegmons, and/or abscesses of the oral mucosa, allergic lesions of the oral mucosa and/or of the tongue, diseases of the upper respiratory tract, nasal sinusitis, allergic rhinitis, allergic rhinopathy, tonsillitis, inflammations and infections of the mucosa of mouth, gingiva, tongue, throat, nose and/or genitals, in particular skin of the penis, of the glans, of the scrotum, of the preputium, cover of the glans, clitoral hood, clitoral glans, outer and inner labia, physical impairments and/or damages of the skin structure comprising injuries, stings, slashes, scratches, abrasions, burns and/or chemical burns, each triggered by contact with at least one biobased, preferably peptidebased, noxa, and/or
inner epithelial tissues (Table 2) comprising diseases of the digestive tract, inflammations of the gastric mucosa and/or small/intestinal mucosa, Morbus Crohn, Colitis ulcerosa, diverticulosis, Claudin-associated diseases, diseases of the lower respiratory tract, inflammations of the lung and/or bronchial tubes, and/or asthma.

Skin lesions comprise skin rednesses (erythemae), skin discolourations, ring-shaped skin alterations, small blisters or blisters (with or without suppuration), pustules, pimples, scabs, raisings, sloughing, etc., strongly itching wheals (e.g. in the form of urticaria) acne, plaques (e.g. psoriasis, psoriasis), ulcers, furuncles, carbuncles, Baghdad boils (skin leishmaniosis, cutaneous leishmaniosis), swelling of the eye lid. The conjunctiva may be inflammatorily altered by different biobased noxae, such as viruses, bacteria, plant noxae, pollen or allergens (see Table 1). The allergic rhinoconjunctivitis is an allergic disease on the conjunctiva.

The epithelial tissues have a reduced selective permeability barrier, among other things based on down-regulated claudin expression, in particular in the case of psoriasis, dermatitis and atopic dermatitis and related skin diseases with barrier impairment, in particular in the early stage.

The afore-mentioned diseases may also occur as occupational health diseases resulting from increased exposure by biobased noxae (Table 1) at the workplace and/or resulting from increased sensibility of human.

In a further embodiment of the use according to the invention of the at least one compatible solute or solute mixture of formula I and/or of formula II, the at least one biobased, preferably peptidebased, noxa intrudes the outer epithelial tissue, in particular the epidermis, preferably the surface epithelium of the skin, the multi-layered epithelium of the oral mucosa, of the tongue mucosa, of the nasal mucosa, of the cornea or conjunctiva of the eye. Modulation, alteration and/or damage of the selective permeability barrier, in particular of the tight junction, by influence of the biobased, preferably peptidebased, noxae may ensue penetration of the noxae at least in the Stratum corneum and at least in part in the Stratum granulosum and/or in deeper epithelial layers.

The compatible solute or solute mixture described afore, preferably ectoine and/or hydroxyectoine, is particularly suitable for use in prevention or treatment of diseases of epithelial tissues (Table2) caused by at least one biobased noxa (Table 1) comprising
(1) infections comprising parasitically, bacterially, virally and/or infections caused by fungi (Table 1). In particular, such infections comprise infections of outer epithelial tissues, such as skin infections comprising erythrasmae, Impetigo contiaguisa (tetter), Herpes labialis, phlegmons, furuncles, skin tuberculosis and Tinea pedis (athlete's foot); infections of transitional epithelial tissues, such as upper respiratory tract and oral cavity, comprising infectious nasal sinusitis, infectious gingiva infections, bacterial periodontitis, of inner epithelial tissues, such as respiratory infections comprising infectious bronchitis, bronchiolitis and/or alveolitis;
(2) allergies triggering an immune response of the body on non-infectious noxae (antigens or allergens, respectively) (Table 1) with inflammatory signs, comprising allergies of outer epithelial tissues, such as urticaria, contact eczemas, neurodermatitis and allergic conjunctivitis; of transitional epithelial tissues, such as Rhinitis allergica, allergic rhinopathy and sinusitis; of inner epithelial tissues, such as Asthma bronchiale, and
(3) mechanical lesions caused by environmental noxae (Table 1), such a high temperature, extreme cold, low humidity and/or wind, comprising dry, rough and/or chapped outer epithelial tissues, such as skin, cornea and/or conjunctiva of the skin, ripped corner of the mouth, chapped lips and sunburn, dry transitional epithelial tissues, such as dry mucosae of the nose. Such mechanical primary interferences of epithelial tissues may already result in damage of the barrier function of the affected epithelial tissues and a secondary damage by further biobased, preferably peptidebased, noxae (Table 1, a, b), c), d) and/or e)) may result.

Within the meaning of the invention, primary interferences and/or damages of the barrier function of epithelial tissues are such interferences and/or damages which occur initially, which inconclusively are phenotypically noticed and which are not pathological. As the case may be, a cosmetic or medical product is suitable for prevention or treatment of such barrier malfunctions. Secondary interferences and/or damages of the barrier function of epithelial tissues may arise out of such primary barrier malfunctions. Within the meaning of the invention, secondary barrier malfunctions are such interferences and/or damages which follow on a primary barrier malfunction and result in further damage of the barrier function, being phenotypically clearly noticeable and characterisable. This is usually the case where environmental noxae cause mechanical lesions according to group (3) described above and subsequently infections are caused by infections noxae (see group (1) above) or allergies are caused by non-infectious noxae (see group (2) above). Usually, secondary barrier malfunction are pathological and have to be treated with a medical product or medicament. Within the meaning of the invention, secondary barrier malfunctions may also occur without primary barrier malfunctions. The symptoms respectively occurring and phenotypically appearances are already described above. The preferred embodiments of the at least one compatible solute, solute mixture and/or compositions as well as concentrations described below apply accordingly for the afore-mentioned groups.

In an embodiment according to the invention, the at least one compound is selected from S-ectoine, R-ectoine, (S,S)-hydroxyectoine, (S,R)-hydroxyectoine, (R,S)-hydroxyectoine, (R,R)-hydroxyectoine and S-homoectoine, physiologically compatible salts of S-ectoine, R-ectoine, (S,S)-hydroxyectoine, (S,R)-hydroxyectoine, (S,R)-hydroxyectoine, (R,S)-hydroxyectoine, (R,S)-hydroxyectoine, (R,R)-hydroxyectoine and S-homoectoine, amides and esters of the afore-mentioned compounds, or is a solute mixture of at least two of the afore-mentioned compounds

A compatible solute or a solute mixture comprising at least two of the mentioned compounds of the preceding definition is preferred, wherein the at least one compound is selected from S-ectoine, R-ectoine, (S,S)-hydroxyectoine, (S,R)-hydroxyectoine, (R,S)-hydroxyectoine, (R,R)-hydroxyectoine, and S-homoectoine, physiologically compatible salts of S-ectoine, R-ectoine, (S,S)-hydroxyectoine, (S,R)-hydroxyectoine, (S,R)-hydroxyectoine, (R,S)-hydroxyectoine, (R,S)-hydroxyectoine, (R,R)-hydroxyectoine and S-homoectoine, amides and esters of the afore-mentioned compounds, or is a solute mixture of at least two of the afore-mentioned compounds. S-enantiomer according to CIP priority rules corresponds to L-enantiomer according to Fisher projection, and R-enantiomer according to CIP priority rules corresponds to D-enantiomer according to Fisher projection.

The afore-mentioned solutes are particularly preferred compounds of formula I and of formula II for use in the prevention or treatment of barrier malfunctions of epithelial tissues associated with at least one biobased noxa, in particular of diseases comprising at least one barrier malfunction in at least one cell layer of at least one epithelial tissue.

In a preferred embodiment of the solute or solute mixture according to the invention for use in the prevention or treatment of barrier malfunctions of epithelial tissues associated with biobased noxa, the at least one compatible solute is present in enantiopure form with a purity of greater than or equal to 90%, preferably greater than or equal to 95%, greater than or equal to 97%, greater than or equal to 99%, particularly preferably equal to 100%. This means that, relating to the solute mixture, the mixture of two compounds has the respective compound in enantionpure form, and preferably has no contamination of the selected compound by its isomer. Enantiopure forms of the solute or solute mixture according to the invention preferably have S- and/or (S,S)-isomers.

Preferred forms of the solute according to the invention include S(L)-ectoine, R(D)-ectoine, (S,S)-hydroxyectoine, (S,R)-hydroxyectoine, (R,S)-hydroxyectoine, and (R,R)-hydroxyectoine, as well as solute mixtures of at least two of the afore-mentioned compounds.

In a preferred enantiopure solute mixture, S-ectoine and (S,S)-hydroxyectoine each are present with a purity of greater than or equal to 90%, greater than or equal to 95%, preferably greater than or equal to 97%, greater than or equal to 99%, particularly preferably equal to 100%. This solute mixture thus preferably has less than or equal to 10%, less than or equal to 5%, preferably less than or equal to 3%, less than or equal to 1%, particularly preferably equal to 0% R-ectoine, or (R,S)-/(S,R)- or (R,R)-hydroxyectoine.

In a special embodiment of the invention, the following racemates are preferred:
S-ectoine and R-ectoine
(S,S)-hydroxyectoine, and (S,R)-hydroxyectoine, (R,S)-hydroxyectoine and (R,R)-hydroxyectoine, or
S-homoectoine and R-homoectoine.

The solute mixture according to the invention comprises at least two compounds according to formula I and/or formula II and/or their respective enantiomers. The solute mixture according to the invention described afore for use in the prevention or treatment of barrier malfunctions of epithelial tissues associated with biobased, preferably peptidebased, noxae comprises, based on the sum of all compounds in the solute mixture having a total content of 100% by weight,
an amount of S-ectoine of greater than or equal to 50% by weight to less than or equal to 100% by weight, and an amount of (S,S)-hydroxyectoine of greater than or equal to 50% by weight to less than or equal to 100% by weight, and preferably,
an amount of S-ectoine of greater than or equal to 75% by weight, preferably greater than or equal to 85% by weight to less than or equal to 95% by weight, and an amount of (S,S)-hydroxyectoine in a range of greater than or equal to 5% by weight to less than or equal to 25% by weight, preferably less than or equal to 15% by weight.

In a special embodiment of the solute mixture according to the invention is
the amount of S-ectoine less than or equal to 70% by weight, preferably less than or equal to 65% by weight, less than or equal to 60% by weight, less than or equal to 55% by weight, particularly preferably greater than or equal to 50% by weight, and
the amount of (S,S)-hydroxyectoine greater than or equal to 30% by weight, preferably greater than or equal to 35% by weight, greater than or equal to 40% by weight, greater than or equal to 45% by weight, particularly preferably less than or equal to 50% by weight.

In an embodiment, the solute mixture comprises a mixture of two compounds of formula I and/or of formula II each having a content of greater than or equal to 60% by weight of the first compound to less than or equal to 40% by weight of the second compound. Preferably, said solute mixture comprises greater than or equal to 60% by weight, particularly preferably greater than or equal to 70% by weight S-ectoine and less than or equal to 40% by weight, particularly preferably less than or equal to 30% by weight (S,S)-hydroxyectoine. In a special embodiment, the solute mixture comprises a mixture of two compounds of formula I and/or of formula II having a content of 50% by weight S-ectoine and 50% by weight (S,S)-hydroxyectoine.

In a special embodiment of the use according to the invention in the prevention or treatment of barrier malfunction of epithelial tissues (Table 2) associated with biobased noxae (Table 1), the at least one solute of formula I and/or of formula II, or the solute mixture comprising at least two solutes of formula I and/or of formula II is biobased and therefore of biological origin.

Within the meaning of the invention, biobased or of biological origin means that the compound of formula I and/or of formula II is produced by or in an organism, respectively. Preferably, the organism is a microorganism and, particularly preferably, the microorganism is a halophilic bacterium comprising Ectothiorhodospira halochloris, Halomonas elongate, Marinococcus halophilus, Brevibacterium linens, Halomonas SPC1, Volcaniella eurihalina, Deleya salina, Bacillus pantothenticus, Bacillus halophilus, Vibrio costicola and Streptomyces parvulust. Within the meaning of the invention, the biobased solute of formula I and/or of formula II, or the solute mixture described above, preferably S-/R-ectoine and/or (S,S)-/(S,R)-/(R,S)-/(R,R)-hydroxyectoine, is produced in Halomonas elongate, Brevibacterium lines or Marinococcus halophilus and is obtained from said bacteria.

Compounds of formula I or of formula II, as well as the solute mixture comprising S-ectoine and (S,S)-/(S,R)-/(R,S)-/(R,R)-hydroxyectoine, being produced in biotechnologically modified organisms, preferably in recombinant microorganism comprising, but not limited to the afore-mentioned strains, are also biobased or of biological origin since the afore-mentioned compounds are produced by equipment of biological cell rather than synthetically in chemistry laboratory without involvement of a biological organism. Compounds of formula I or of formula II, having identical structure, being synthetically produced outside of an organism described before, are not included by the definition of biobased solutes.

A further subject matter of the present invention is a pharmaceutical composition comprising at least one compatible solute, in particular in the manner described afore, or one solute mixture comprising at least two compatible solutes for use in the prevention or treatment of barrier malfunctions of epithelial tissues associated with at least one biobased, preferably peptidebased, noxa (Table 1), in particular diseases comprising at least one barrier defect, prefearbly in at least one cell layer, in at least one epithelial tissue. In this context, the compatible solute or the solute mixture comprises at least one compound selected from compounds of formula I, of formula II, physiologically compatible salts of formula I and formula II, stereoisomeric forms of the compounds of formula I, formula II, and physiologically compatible salts of the stereoisomeric forms, or from a mixture of at least two of the afore-mentioned compounds. Definition of formula I and formula II as well as of their residues R1, R2, R3, R4 and R5 as well as n and alkyl are already defined above. The definitions and preferred residues, preferred compounds and combinations apply for the composition accordingly. S(L)-ectoine, R(D)-ectoine, (S,S)-hydroxyectoine, (S,R)-hydroxyectoine, (R,S)-hydroxyectoine and/or (R,R)-hydroxyectoine are particular preferred.

In a further embodiment of the use according to the invention of the afore-mentioned composition, the at least one compatible solute, preferably ectoine and/or hydroxyectoine, or the solute mixture is present in the composition in an amount of greater than or equal to 0.0001% by weight to less than or equal to 50% by weight, based on the total content of the composition.

Preferred compositions comprise the at least one compatible solute or the solute mixture, preferably S(L)-ectoine, R(D)-ectoine, (S,S)-hydroxyectoine, (S,R)-hydroxyectoine, (R,S)-hydroxyectoine and/or (R,R)-hydroxyectoine, in the composition in an amount of greater than or equal to 0.0001% by weight to less than or equal to 50% by weight, based on the total content of the composition. A range greater than or equal to 0.001% by weight, greater than or equal to 0.01% by weight, greater than or equal to 0.1% by weight, particularly preferably greater than or equal to 1.0% by weight to less than or equal to 40% by weight, less than or equal to 30% by weight, less than or equal to 20% by weight, particularly preferably less than or equal to 10% by weight, is particularly preferred.

In a further embodiment of the use according to the invention of the afore-mentioned composition, the at least one compatible solute or the solute mixture, preferably S(L)-ectoine, R(D)-ectoine, (S,S)-hydroxyectoine, (S,R)-hydroxyectoine, (R,S)-hydroxyectoine and/or (R,R)-hydroxyectoine, is present in the composition in an amount of greater than or equal to 0.0001% by weight to less than or equal to 10% by weight, based on the total content of the composition.

In a particularly preferred composition according to the invention, the at least one compatible solute or the solute mixture is present in the composition in an amount of greater than or equal to 00001% by weight, to less than or equal to 10% by weight, based on the total content of the composition, preferably greater than or equal to 0.001% by weight to less than or equal to 8% by weight, preferably to less than or equal to 6% by weight, particularly preferably less than or equal to 5% by weight.

The composition according to the invention preferably comprises at least one compound of formula I and/or of formula II selected from S-ectoine, R-ectoine, (S,S)-hydroxyectoine, (S,R)-hydroxyectoine, (R,S)-hydroxyectoine, (R,R)-hydroxyectoine and S-homoectoine, physiologically compatible salts of S-ectoine, R-ectoine, (S,S)-hydroxyectoine, (S,R)-hydroxyectoine, (S,R)-hydroxyectoine, (R,S)-hydroxyectoine, (R,S)-hydroxyectoine, (R,R)-hydroxyectoine and S-homoectoine, amides and esters of the afore-mentioned compounds, or is a solute mixture of at least two of the afore-mentioned compounds. Particularly preferably, at least one of the afore-mentioned compatible solutes or the solute mixture is present in the composition in an amount of greater than or equal to 0.0001% by weight to less than or equal to 10% by weight, based on the total content of the composition.

In a further embodiment of the use according to the invention of the afore-mentioned composition and/or of the solute or solute mixture according to the invention, each in particular for use in prevention or treatment of barrier malfunction of epithelial tissues associated with at least one biobased noxa, the solute, solute mixture and/or the compositions is present as medicament, medical product, cosmetic, as addition to one of the afore-mentioned products and/or preferably as component of in-vitro diagnostic products (IVD). The solute or the solute mixture may be admixed to recipes and/or formulations of existing medicaments to add the stabilising, protective (preventive) and/or supporting (therapeutic) effectiveness of the solute on the barrier function of epithelial tissues.

In a further embodiment of the use according to the invention of the afore-mentioned compositions and/or of the solute or solute mixture according to the invention each for use in the prevention or treatment of barrier malfunctions of epithelial tissues associated with at least one biobased noxa, the solute, solute mixture or the composition is present in solid or liquid form, selected from
i) solid forms comprising powder, lyophilisate, tablets, granule, form-coated tablet, dragee, capsules, effervescent tablets, powder, and soap,
ii) liquid forms comprising solution, injection, infusion, tincture, infusion solution, suspension, emulsion, application, foam, and cream, and/or
iii) mixtures comprising spray, aerosols, ointment, paste and capsule, and in particular semi-solid forms.

Preferably, these forms preferably comprise at least one compatible solute of formula I and/or of formula II, preferably S-ectoine and/or (S,S)-hydroxyectoine, particularly preferred having an amount of greater than or equal to 0.0001% by weight to less than or equal to 10% by weight in the composition, based on the total content of the composition.

For oral administration for ingestion via gastrointestinal tract by swallowing of the composition according to the invention and preferably for use in the prevention or treatment of diseases of the inner epithelial tissues of gastric and/or intestinal mucosa comprising at least one barrier malfunction, are particularly preferred
i) solid forms comprising powder, in particular after mixing with a beverage or water, capsules, tablets, granule, film-coated tablet, dragee, and effervescent tablets, or
ii) liquid forms comprising solution, infusion solution, tincture, syrup, juice, and oil.

Liquid formulations, preferably solutions, tinctures, rinses, solutions for gargling and infusion solutions and/or mixtures, such as sprays, are preferably used for use in prevention or treatment of diseases of transitional epithelial tissues of the oral cavity, nasal cavity, throat and palate comprising at least one barrier malfunction respectively.

Solid forms, such as powder, powder, soap or mixtures, such as ointments, creams, applications, hydrogels, are prefearbly used for use in prevention or treatment of diseases of outer epithelial tissues of skin, outer skin, lips and outer ear, comprising at least one barrier malfunction respectively.

Each composition according to the invention may comprise excipients known from the state of the art in the selected formulation. Excipients comprise carriers, preservatives, antioxidants, stabilisers, solubilisers, vitamins, colourants, smell improving agents.

Carriers, in particular for cosmetic formulations and/or medical products, comprise animal and vegetable fats, waxes, paraffins, starch, tragacanth, cellulose derivatives, polyethylene glycols, silicons, bentonites, silicic acid, talcum, and zinc oxide, or mixtures of at least two of the afore-mentioned substances. The afore-mentioned carriers are particularly well-suited for ointments, pastes, creams and applications comprising at least one solute of formula I or of formula II, preferably S-ectoine and/or (S,S)-hydroxyectoine.

Carriers for powders or sprays comprising at least one solute of formula I or of formula II, preferably S-ectoine and/or (S,S)-hydroxyectoine comprise lactose, talcum, silicic acid, aluminium hydroxide, calcium silicate and polyamide powder, or mixtures of at least two of the afore-mentioned substances. Sprays can additionally comprise common propellants, for example hydrochlorfluorocarbons, propane/butane or dimethylether.

Solutions and emulsions comprising at least one solute of formula I or of formula II, preferably S-ectoine and/or (S,S)-hydroxyectoine, can comprise usual carriers, such as solvents, solubilizers and emulsifiers, e.g. water, ethanol isopropanol, ethylcarbonate, ethylacetate, benzylalcohol, benzylbenzoate, propylene glycol, 1,3-butylglycol, oils, in particular cottonseed oil, peanut oil, maize germ oil, olive oil, castor oil and sesame oil, glycerin fatty acid ester, polyethylene glycol and fatty acid esters of sorbitan, or mixtures of at least two of the afore-mentioned substances.

Suspensions comprising at least one solute of formula I or of formula II, preferably S-ectoine and/or (S,S)-hydroxyectoine, comprise usual carriers, such as liquid diluents, e.g. water, ethanol or propylene glycol, suspension agents, e.g. ethoxylated isosterylalcohols, polyoxymethylene sorbitol ester and polyoxymethylene sorbitan ester, microcrystalline cellulose, aluminium metahydroxide, bentonite, agar agar and tragacanth, or mixtures of at least two of the afore-mentioned substances.

Soaps and lotions comprising at least one solute of formula I and or of formula II, preferably S-ectoine and/or (S,S)-hydroxyectoine, which can comprise carriers, such as alkali salts of fatty acids, salts of fatty acid semi-esters, fatty acid protein hydrolysate, isothionates, lanolin, fatty alcohol, vegetable oils, botanical extracts, glycerin, sugar, or mixtures of at least two of the afore-mentioned substances, are particularly well-suited for daily use. Further excipients being suitable for the respective formulation are known by the person skilled in the art.

For prevention or treatment of barrier malfunctions of epithelial tissues associated with at least one biobased noxa, in particular diseases comprising at least one barrier malfunction, are used
a) cosmetic formulations comprising mixtures selected from lipstick, lip balm, powder, sun milk, body lotion, and/or
b) pharmaceutical compositions (medicaments) medical products or IVD, preferably comprising S-ectoine and/or (S,S) hydroxyectoine, in solid or liquid form, wherein the formulation is selected from
   i) solid forms comprising powder, tablets, granule, film-coated tablet, dragee, capsules, effervescent tablets, powder, and soap,
   ii) liquid forms comprising solution, injection, infusion, tincture, infusion solution, suspension, syrup, juice, emulsion, application, foam, cream, lotion, surfactant-containing cleaning preparation, oil, and/or
   iii) mixtures comprising spray, aerosols, inhalant, ointment, paste, applications and hydrogels.

Preferred formulations according to the invention, in particular medical products, for prevention or treatment of barrier malfunctions of epithelial tissues associated with at least one biobased noxa, preferably comprising S-ectoine and/or (S,S)-hydroxyectoine, are suitable formulations for topical administration on the skin surface, on the eye, on the oral and nasal mucosa. They particularly preferably comprise S-ectoine and/or (S,S)-hydroxyectoine, being in the composition in an amount of greater than or equal to 0.0001% by weight to less than or equal to 10% by weight, based on the total content of the composition.

Within the meaning of the invention, medical products include the formulations described herein comprising at least one solute of formula I or of formula II or the solute mixture of at least two of the afore-mentioned compounds, preferably S-ectoine and/or (S,S)-hydroxyectoine, for use in the prevention or treatment of human in medical therapeutic purposes. Preferably, the medical products according to the invention meet the specification and requirements of guideline 93/42/EWG and/or of appropriate regulations in the USA.

Within the meaning of the invention, in-vitro diagnostic products (IVD) comprise at least one solute of formula I or of formula II or the solute mixture from at least two of the afore-mentioned compounds as additive (synonymously component) and preferably meet the specification and requirements of guideline 98/79/EG and/or regulations in the USA “U.S. gouvernement regulations Title 21: Food and Drugs PART 809—IN VITRO DIAGNOSTIC PRODUCTS FOR HUMAN USE Subpart A—§ 809.3 Definitions”.

Preferred excipients for compositions of medical products and pharmaceutical formulations in solid or liquid form for prevention or treatment of diseases comprising at least one barrier malfunction in epithelial tissues comprise lactose, sucrose, dextrose, mannitol, sorbitol, starch, gelatine, tragacanth, pectin, cellulose, methylcellulose, hydroxypropylmethylcellulose (HPMC), carboxymethylcellulose sodium, ethylcellulose, hydroxypropylmethylcellulose phthalate, cellulose acetate phthalate, polyvinylpyrrolidone, polyvinylalcohole, polyacrylic acid, polyethylene glycol, polyethylen oxide, sodiumdodecylsulfate, sodiumacetylstearylsulfate, and sodiumdioctylsulfosuccinate (also K salts, Ca salts).

Preferred excipients for solutions and suspensions according to the invention comprise dextrose, mannitol, tragacanth, pectin, methylcellulose, hydroxypropylmethylcellulose (HPMC), carboxymethylcellulose sodium, polyvinylpyrrolidone, polyvinylalcohole, polyacrylic acid, polyethylene glycol, polyethylene oxide, sodiumdodecylsulfate, sodiumacetylstearylsulfate, sodiumdioctylsulfosuccinate (also K salts, Ca salts), and, in particular for suspensions, cellulose.

Within the meaning of the invention, semi-solid forms or mixtures preferably comprise hydroxypropylmethylcellulose (HPMC), carboxymethylcellulose sodium, polyethylene glycol, polyethylene oxide, sodiumdodecylsulfate, sodiumacetylstearylsulfate and pectin.

A preferred composition of a medical product and/or of a medicament comprising the solute according to the invention of formula I or of formula II or the solute mixture of at least two of the afore-mentioned solutes, preferably S-ectoine and/or (S,S)-hydroxyectoine, for prevention or treatment of barrier malfunction in epithelial tissues associated with at least one biobased noxa comprises formulations for applying, depositing, massaging in, spraying on and/or laying on the affected epithelial surface. Such products comprise nasal spray, nose drops, nasal balm, eye drops, artificial tear supplement (dry eyes), contact lenses, eye pads (dressings), eye gel, mouth wash, oral spray, gels for the gingiva, ear drops, solution, rinse, suspension, ointment, cream, lotion, paste, spray, jelly, aerosols, emulsions (W/O, O/W), hydrogel, liposomes, microsomal capsules, vapour bath concentrate. Dermatological products are particularly preferred for prevention or treatment of diseases comprising at least one barrier malfunction of outer epithelial tissues and/or transitional epithelial tissues. The preferred combinations of features described afore apply for the mentioned products accordingly.

### DESCRIPTION OF THE FIGURES (FIG.)

FIG. 1 Experimental setup for determination of the transepithelial resistance (TEER); An insert (3) having a transparent ThinCerts-membrane, pore size 0.4 μm, is arranged in the experimental vessel (1) with a cell culture medium (2). A single-layered cell layer of a cell culture (4) is present on the membrane, wherein the membrane with the cells is covered by the cell culture medium. The single-layered cell layer (4) is arranged between the two electrodes E1 and E2, wherein a defined strength (U) of a direct current (DC) is applied between the electrodes E1 and E2 such that the applied direct current (DC) flows through the cells.

FIG. 2 TEER-values [Ohm] of epithelial cells of oral mucosa cells (TR146) after incubation with 50 mM ectoine, 0.01% lecithin or PBS for 12 hours (h).

FIG. 3 TEER-values [Ohm] of epithelial cells of oral mucosa cells (TR146) after incubation with 50 mM ectoine, 0.01% lecithin or PBS over night and subsequent treatment with 0.005% SDS.

FIG. 4 TEER-values [Ohm] of renal porcine epithelial cells (LLC-PK1) after incubation with 50 mM ectoine, 0.01% lecithin or PBS over night and subsequent treatment with 0.001% BAK.

FIG. 5 TEER-values [Ohm] of human keratinocytes (HaCat) after incubation with 50 mM or 100 mM ectoine or PBS over night and subsequent drying for 5 min at room temperature (RT).

FIG. 6 Experimental setup to the allergy prevention assay (APA): An insert (1) with a transparent ThinCerts-membrane (3) having a pore size of 0.4 μm is arranged in an outer reservoir (5) in an experimental vessel (6) with cell culture medium. A closed single-layered epithelial cell layer (2) lays on the membrane (3). The insert partitions off an inner reservoir (7) above the cell layer (3). The allergen is e.g. OVA.

The following examples show the influence of ectoine on different epithelial tissues and on the barrier function of epithelial cells and epithelial tissues, respectively. It is shown for ectoine to be effective in prevention and treatment of barrier malfunctions of epithelial tissues associated with at least one biobased noxa. Ectoine structurally acts on epithelial tissues.

### EXAMPLES

Theoretical Background on the Barrier Function of Epithelial Tissues

Diffusion barrier or membrane integrity, respectively, is characterised by epithelial cells as well as endothelial cells which have intercellular junctions. These intercellular junctions separate the apical (lumen) side from the basolateral (ablumen) side of the cell and form a diffusion barrier due to their complex geometry for a large number of molecules in the paracellular passage between the apical (luminal) and basolateral (abluminal) compartment as well as for the passage via the intracellular transport path.

This diffusion barrier is ensured by so-called tight junctions (intercellular junctions) connecting adjacent cells with each other (Abbott N J, Ronnback L, Hansson E (2006) Astrocyte-endothelial interactions at the blood-brain barrier. Nat Rev Neurosci, 7: 41-53). Tight junctions have a complex structure and consist of a variety of components comprising integral membrane proteins (claudins, occludins and junction adhesion molecules “JAMs”) and peripheral membrane proteins. The claudin protein family up to now comprises 24 described members of different cell types. Of these, 21 are known to be components of tight junctions in epithelial membranes of kidney, liver, brain and intestinal tissue. Claudins are to be found in homotypical as well as heterotypical arrangements in several tight junctions and may be divided into two main categories: Pore-sealing (“pore-sealing”) and pore-forming (pore-forming) Claudins. Claudin-1, -3, -4, -5, -7 and -19 are known to be pore-sealing (“pore-sealing”) Claudins. An enhanced expression of pore-sealing claudins results in an increase of the density of epithelial tissues, and in an enhanced transepithelial electric resistance (TEER) and in a decreasing permeability of epithelial tissues (Khan N, Asif, AR (2015) Transcriptional Regulators of Claudins in Epithelial Tight Junctions. Mediators Inflamm, Volume 2015, Article ID 219843, doi: 10.1155/2015/219843). On the basis of this systematics, the experiments for proof of the effects of ectoine on epithelial tissues were performed.

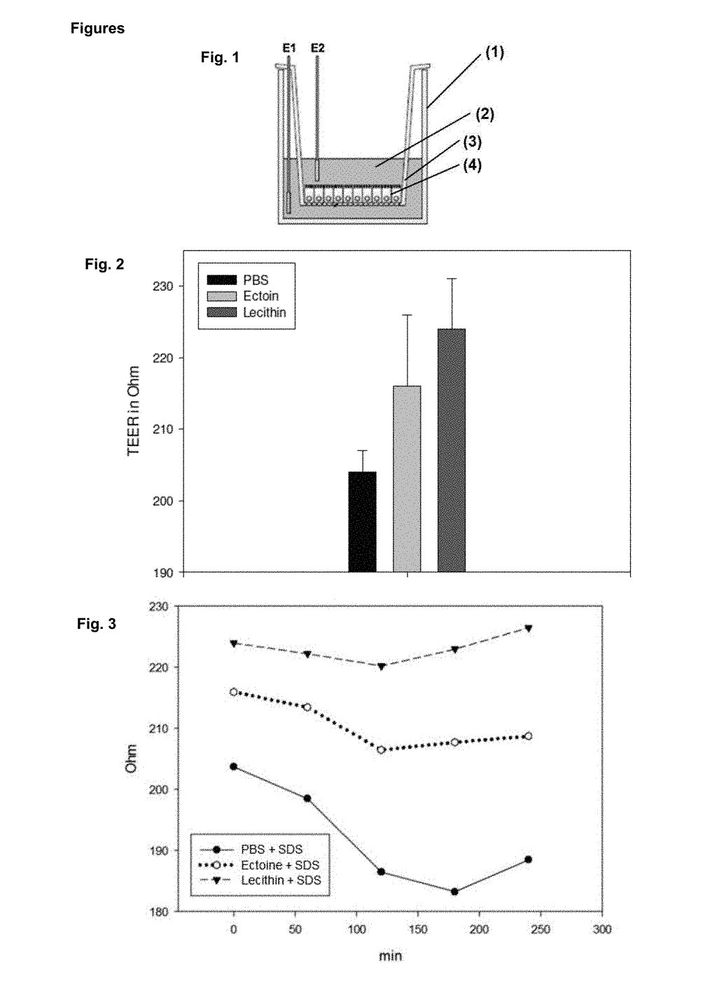

An intact diffusion barrier or the determination of the integrity of epithelial cells or endothelial cells, respectively, may be measured in a number of ways. The OECD Guidance Document 28 suggests the determination e.g. by means of transepithelial electric resistance (TEER), as shown in FIG. 1.

The addition of some compounds, e.g. hydrocortisone or the phospholipid lecithin, to a cell culture or to epithelial cells, respectively, strengthens the membrane integrity being measurable on the basis of an enhanced electric resistance (TEER) in [Ohm]. However, there are a variety of components which weakens the tight junction wherein the membrane integrity decreases (Wegener J, Abrams D, Willenbrink W, Galla H J, Janshoff A (2004) Automated multi-well device to measure transepithelial electrical resistance Under physiological conditions. Biotechniques, 37: 590).

Material

Cell Culture Medium:
MEM medium, PAN-Biotech
DMEM medium (high glucose), PAN-Biotech
HAMS-F12, PAN-Biotech
medium 199, PAN-Biotech

TABLE 3
Chemicals and cell cultures used

| Abbreviation | Explanation | Origin |
|---|---|---|
| PBS | phosphate buffer | PAN Biotech |
| TR146 | human oral mucosa epithelial cells | Sigma |
| LLC-PK1 | porcine renale epithelial cells | DSMZ |
| SIRC | rabbit corneal epithelial cells | LGC |
| HaCaT | human keratinocytes cell line | Uni Münster |
| RPMI-2650 | human nasal mucosa epithelial cells | CLS |
| RLE | rat bronchial epithelial cells (lung) | IUF Düsseldorf |
| SDS | sodium dodecyl sulfate, detergent | Sigma |
| BAK | benzalkonium chloride, preservative | Sigma |
| OVA | ovalbumin | Hyglos |
| Lecithin | positive control for TEER assay | Sigma |
| Claudin-1 ELISA | representative protein of tight junction | Antibodies-Online |
| Ectoin-D | compatible solute according to the invention | bitop AG |
| Ectoine-D | according to formula I or II | |
| OVA ELISA | ovalbumin (native) ELISA kit (Agro-Bio), | Antibodies-Online |
| | representative of an allergen | |

### Example 1

### Impact of Compatible Solutes on Transepithelial Electric Resistance (TEER) of Epithelial Tissues

1.1 Method

The experimental setup is shown in FIG. 1. A defined strength (U) of a direct current (DC) is applied between two electrodes E1 and E2 for determination of TEER, wherein the single-layered cell layer (monolayer) is arranged between the two electrodes (FIG. 1), such that the applied direct current (DC) flows through the cells. The measurement of TEER is made by an epithelial voltmeter (EVOM2). The resulting current I is measured according to the ohmic resistance R, wherein R=U/I (Benson K, Cramer S, Galla H J (2013) Impedance-based cell monitoring: Barrier properties and beyond. FluidsBarriers, 10:5.).

A decrease of the transepithelial electric resistance [Ohm] indicates a defective barrier function of epithelial cells and thus lower membrane integrity. In this way, within the meaning of the invention, evidence about the damaging impact of a stress and the protective effectiveness of a compatible solute may be shown in a comparison between untreated epithelial cells (PBS) and treated epithelial cells (SDS, BAK, air drying each with or without ectoine).

For this purpose, in in vitro experiments, epithelial tissues were sown on special cell culture carrier having a membrane (ThinCerts, pore size 0.4 μm) and cultured to a closed, single-layered cell layer (synonymously epithelial monolayer) on the membrane (FIG. 1).

The intact epithelial monolayer was respectively controlled by a TEER measurement. If no increase were measured in the TEER over two days, the epithelial cells have formed an intact epithelial monolayer having intact epithelial barrier (selective permeability barrier).

In order to measure effects of ectoine on barrier function, in particular membrane integrity, TR146 cells of an intact epithelial monolayer were incubated in cell culture medium with PBS (control), with 50 mM ectoine and with 0.1% lecithin (positive control) for 12 hours (h). Directly after incubation, the TEER was measured, as shown in FIG. 2.

In order to subsequently measure the effect of ectoine on membrane stability under the influence of chemicals, an intact epithelial monolayer of epithelial cells of the oral mucosa (TR146) were incubated in cell culture medium with PBS (control), with 50 mM ectoine and with 0.1% lecithin (positive control) over night. Subsequently, stress treatment was made with 0.005% SDS and change of the transepithelial electric resistance [Ohm] was monitored hourly (FIG. 3).

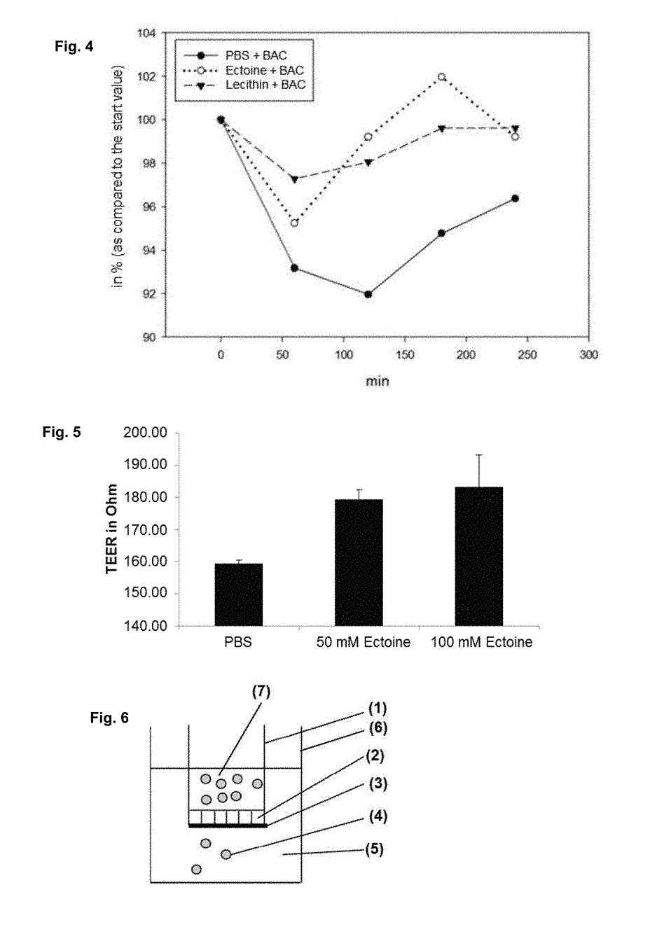

Additionally, an intact epithelial monolayer of porcine renal epithelial cells (LLC-PK1) was also incubated in cell culture medium with PBS (control), with 50 mM ectoine and with 0.1% lecithin (positive control) over night. Subsequently, stress treatment was made with 0.001% BAK and change of the transepithelial electric resistance [Ohm] was monitored hourly (FIG. 4).

In a further experiment, an intact epithelial monolayer of human keratinocytes (HaCaT) was also incubated in cell culture medium with PBS (control), with 50 mM ectoine and with 100 mM ectoine over night. Subsequently stress treatment was made by air drying at room temperature (RT) under the sterile bench for 5 min (FIG. 5). After air drying at RT under sterile conditions, the cells were overlaid with medium including OVA allergen (250 μg/ml) and incubated at 37° C. in the incubator for 24 hours and subsequently the TEER value was measured.

1.2 Results

Measurement of TEER is a direct way to get evidence about the functionality of the barrier of epithelial tissues, in particular concerning membrane integrity and stability. A decrease of the TEER directly indicates an impaired membrane, in particular defective barrier. Chemicals, such as SDS and BAK are able to destroy the membrane, being shown in decreasing TEER values. In the case of too high concentrations of these chemicals, the affected epithelial cells may not recover and die off. The in-vitro experiments described afore proof a protective and stabilizing effect of ectoine on membrane stability or on barrier function of epithelial tissues, respectively, on the basis of TEER values of different epithelial cell lines.

After incubation with the compatible solute, e.g. ectoine, the TEER values [Ohm] were respectively higher than the TEER values of the control without ectoine (PBS). Additionally, the experiments show a protective effect of ectoine against detergents and preservatives.

FIG. 2 already clearly shows the positive effect of ectoine as compared to PBS in non-damaged epithelial cells. Whereas epithelial oral mucosa cells TR146 have a low TEER value of approximately 205 Ohm in PBS, a medium TEER value of 215 Ohm is to be measured in oral mucosa cells after incubation with 50 mM ectoine. The highest TEER value with 225 Ohm is measured for 0.1% lecithin as positive control. It is thus shown that compatible solutes, in particular ectoine and its derivative, additionally stabilize the epithelial membrane.

The addition of membrane-destroying chemicals such as SDS results in strong decrease of membrane stability, being apparent in strong decrease of the TEER values. In this case, addition of 0.005% SDS results in strongest damage of epithelial oral mucosa cells TR146 and in strongest decrease of TEER values to 185 Ohm.

In contrast, incubation with ectoine or lecithin (positive control) prevents damage by SDS. The epithelial cells show with 225 Ohm for lecithin and 215 Ohm for ectoine a comparable TEER and also an intact barrier could be proved as compared to SDS-damaged cells (185 Ohm). Thus, a stabilizing effect on epithelial membrane of oral mucosa cells TRT146 is achieved by ectoine (FIG. 3). In particular, a preventive and thus protective effect of ectoine on the membrane of epithelial cells of the oral mucosa TR146 is proved by pre-incubation with ectoine.

Appropriate results were shown in FIG. 4i n in-vitro experiments with damage of porcine epithelial cells (LLC-PK1) by 0.0001% BAK. Analogously to FIG. 3, the epithelial cells LLC-PK1 show the strongest decrease in TEER [Ohm] and thus the strongest damage of membrane stability (FIG. 5) in the case of damage by BAK (PBS) without ectoine or lecithin (positive control). IN contrast, stabilization of the membrane of epithelial cells LLC-PK1 is already achieved by addition of ectoine.

In a further in-vitro experiment, the epithelial monolayer of keratinocytes (HaCaT) initially obtained was incubated without (PBS) or with ectoine (PBS+ectoine 50/100 mM) and subsequently stressed by air drying. Then, rehydrogenation of the dried keratinocytes with PBS, PBS+ectoine 50 mM or PBS+ectoine 100 mM was made. FIG. 5 clearly shows that drying stress results in damage (TEER=approx. 160 Ohm) of the membrane stability despite rehydrogenation. By comparison, preincubation of keratinocytes with ectoine (TEER=approx. 180 Ohm) obtains better membrane stability or recovers the barrier function of epithelial cells, respectively. These experiments proof a preventive and therapeutic effect of ectoine on the barrier function of epithelial membranes and thus on membrane stability (FIG. 5).

A stabilising effect of ectoine could respectively be identified on the TEER in all experiments for different epithelial cell lines, such as human epithelial cells of oral mucosa TR146, porcine epithelial cells LLC-PK1 and human keratinocytes HaCaT. Thus also a positive effect of ectoine on the barrier function of epithelial membranes. Damage by chemicals (SDS, BAK) and by physical stress (air) could be prevented by ectoine. Preincubation of the respective epithelial cells with ectoine thus proves a preventive effect of ectoine. Additionally, an effect of ectoine recovering membrane stability and barrier function is also proved by addition of ectoine after stress impact (FIG. 5). Appropriate results are to be expected in the case of stress and damage by biobased noxae (Table 1).

As a result, within the meaning of the invention, ectoine is suitable for both prevention and treatment of barrier malfunctions by dryness and/or by allergens (FIG. 2 to FIG. 5).

### Example 2

### Impact of Compatible Solutes on the Penetration of Epithelial Rissues with Allergens—Allergy Prevention Assay (APA)

2.1 Method

The experimental setup is shown in FIG. 6. The cell culture vessel (6) comprises an inner (7) and an outer (5) reservoir, wherein the two compartment are partitioned by a transparent ThinCerts membrane (5) having a pore size of 0.4 μm. This pore size allows for cytokine and protein movement between the outer and inner reservoir (FIG. 6.) Eukaryotic cells may not pass through this membrane thus forming a single-layered closed cell layer which covers the whole membrane. In doing so, an arrangement is achieved by the experimental setup which reflects the apical side (outer reservoir) and basolateral side (inner reservoir within the inset) of epithelial cells. However, allergens, such as e.g. ovalbumin (OVA), may pass the membrane without problems due to their low molecule size.

If a closed epithelial cell layer is present, OVA may only pass this membrane barrier through “gaps”, tight junction or transcytosis from the apical to the basolateral side. (Ding L, Zhang Y, Jiang Y, Wang L, Liu B, Liu J (2014) Transport of egg white ACE-Inhibitory peptide, Gln-Ile-Gly-Leu-Phe, in human intestinal Caco-2 cell monolayers with cytoprotective effect. J Agric Food Chem (Epub ahead of print)).

2.1.1 Method without Drying Stress

The cells of the respective cell line (see Table 3) were cultivated on a membrane (3) (FIG. 6) until formation of a closed single-layered cell layer (2) (synonymously epithelial monolayer). Subsequently, the epithelial monolayer was pretreated with ectoine (10, 50, 100 mM) in cell culture medium for 6 hours. After pretreatment with ectoine, the epithelia monolayer was incubated with 250 μg/ml of the allergen OVA (FIG. 6) over night. Subsequently, the OVA content was measured in the inner and outer reservoir by means of OVA-specific ELISA and the relative penetration of the epithelial monolayer with OVA in pretreated and non-treated cells was determined.

2.1.2 Method with Drying Stress

In order to imitate dry skin, the epithelial monolayer on the membrane was stressed by air drying and fluid loss for 5 minutes. On that account, the cell culture supernatant was removed and the epithelial monolayer was diagonally fixed in a new 12-well plate such that the fluid could flow off. After drying the epithelial monolayer was again transferred into the cell culture medium and treated with ectoine and allergen analogously to 2.1.1.

2.2 Results

2.1.1 Penetration of the Membrane with the Allergen

Penetration of the epithelial monolayer in-vitro with the allergen OVA each with or without ectoine pretreatment was analysed by means of OVA-specific ELISA. Since a considerably smaller amount of OVA was detected in the inner reservoir (basolateral side) after pretreatment with ectoine than in the inner reservoir without pretreatment with ectoine, ectoine pretreatment results in a lower penetration of the epithelial monolayer with OVA as compared to the epithelial monolayer without ectoine pretreatment. As a result, an effect stabilising the permeability barrier of epithelial tissues was proved for ectoine in different cell lines.

In total, five different cell lines were analysed representing different body positions or organs, respectively, which make contact with allergens, e.g. epithelial cells of the oral mucosa (TR146), epithelial cells of the nasal mucosa (RPMI-2650), bronchial epithelial cells (RLE), cornea of the eye (SIRC) and outer skin (HaCaT). A protective effect of ectoine on the barrier function of epithelial tissues could be proved in all tested cell line (Table 4.)

Table 4 shows that the permeability of the epithelial monolayer for OVA is about 40% to 60 lower after pretreatment with 50 mM and 100 mM ectoine as compared to penetration with OVA of the respective epithelial monolayer of the oral mucosa (TR146) in PBS without ectoine pretreatment. In the case of 10 mM ectoine, the effect of ectoine protecting the permeability barrier is only significantly measurable in epithelial monolayer of the oral mucosa (TR146) and of the bronchial tubes (RLE). In the remaining epithelial cell lines, only a very low or no significant stabilising effect could be detected in this experimental setup by means of ELISA for pretreatment with 10 mM ectoine for 6 hours as compared to non-treated cells (PBS).

Considerable effects are to be seen in the case of pretreatment of the epithelial monolayer with 50 mM ectoine. In this case, with the exception of RLE cells, at least 20% to 50% of the allergen OVA is prevented from penetration and permeation of the epithelial membrane from the apical side to the basolateral side by the influence of ectoine (Table 4). In the case of SIRC-, HaCaT- and RPMI-cells each having been pretreated with 100 mM ectoine, up to 50% of the allergen OVA are also retained and do not get to the basolateral side of the epithelial monolayer.

Thus, it has been shown for ectoine to be able to stabilise membrane structure and barrier functions of epithelial tissues and to inhibit penetration of allergens.

2.2.2 Penetration of the Membrane with Allergens After Drying Stress

In a further in vitro experiment, OVA penetration of dried epithelial cells was measured by means of ELISA according to 2.1.1. It has been shown that OVA could penetrate to the basolateral side (lumen) without the addition of ectoine (PBS) (Table 4) after drying and rehydrogenation of the HaCaT epithelial monolayer. In contrast, pretreatment with 50 mM ectoine already results in recovery of the membrane barrier such that inhibition of penetration of OVA by 12% is achieved by 50 mM ectoine. By comparison, inhibition of OVA penetration by 55% was achieved with 50 mM ectoine in the case of HaCaT epithelial monolayer without drying stress (Table 4).

Thus, it could be clearly shown in five different epithelial cell lines that ectoine stabilises the cell membrane. Penetration of the epithelial cell layer with noxae could be inhibited by ectoine in both the skin and in the oral mucosa. The barrier function of the whole cell layer is strengthened against penetration of allergens by stabilisation of the membrane. Thus, ectoine has protecting effect.

TABLE 4
Relative penetration protection against the allergen OVA

| | | Penetrations protection |
|---|---|---|
| Epithelial monolayer | | (as compared to PBS) |
| of cell culture | Treatment | [%] +/− SD |
| SIRC | OVA + PBS | 0 +/− 5.64 |
| | OVA + 10 mM ectoine | 0 +/− 11.52 |
| | OVA + 50 mM ectoine | 16.3 +/− 2.31 |
| | OVA + 100 mM ectoine | 29.5 +/− 16.99 |
| HaCaT | OVA + PBS | 0 +/− 13.33 |
| | OVA + 10 mM ectoine | −0.61 +/− 0.1 |
| | OVA + 50 mM ectoine | 43.5 +/− 13.01 |
| | OVA + 100 mM ectoine | 52.4 +/ 10.44 |
| TR146 | OVA + PBS | 0 +/− 7.01 |
| | OVA + 10 mM ectoine | 12.07 +/ 2.31 |
| | OVA + 50 mM ectoine | 32.52 +/ 10.01 |
| | OVA + 100 mM ectoine | 13.44 +/− 7.69 |
| RPMI | OVA + PBS | 0 +/ 11.1 |
| | OVA + 10 mM ectoine | 23.5 +/− 17.1 |
| | OVA + 50 mM ectoine | 69.3 +/− 35.4 |
| | OVA + 100 mM ectoine | 67.1 +/− 14.1 |
| RLE | OVA + PBS | 0 +/− 2.45 |
| | OVA + 10 mM ectoine | 12.7 +/− 10.4 |
| | OVA + 50 mM ectoine | −6.3 +/−7.91 |
| | OVA + 100 mM ectoine | −124.5 +/− 3.72 |
| HaCaT (drying stress) | OVA + PBS | 0 +/− 7.36 |
| | OVA + 50 mM ectoine | 8.1 +/− 8.01 |
| | OVA + 100 mM ectoine | 11.6 +/− 6.25 |

The experiments clearly show a preventive effect for ectoine (Table 4). Additionally, a therapeutic effect was shown since actually the barrier was partially recovered again in the case of dry skin.

As a result, within the meaning of the invention, ectoine and its derivatives are suitable as compatible solutes for both use in the prevention and treatment of barrier malfunctions in epithelial tissues, in particular of diseases with at least one barrier malfunction in at least one cell layer of at least one epithelial tissue. Even if these experiments were presently not performed, it is to be expected that the results will be confirmed by experiments with artificial skin models (e.g. phenion model, Henkel) or in animal experiments.

### Example 3

### Impact of Compatible Solutes on Tight Junction—Claudin-1 Expression

3.1 Method

The HaCaT cell line was cultured in a plastic cell culture vessel until a closed single-layered cell layer (HaCaT monolayer) was formed. Subsequently, the monolayer was pretreated with 50 mM or 100 mM ectoine in cell culture medium for 6 hours (h). After pretreatment, temperature treatment of the cells was made at 44° C. for 30 min. After heat stress, the monolayer was again incubated in the incubator at 37° C. for 24 hours. Subsequently, the HaCaT monolayer was harvested and, after lysis by means of cryoshock (freezing, unfreezing and scraping with spatula), the lysate was analysed for Claudin-1 by means of ELISA.

3.2 Results

Significant increase of Claudin-1 expression in human keratinocytes (HaCaT) was detected after 24 hours incubation with 100 mM ectoine. By comparison, after incubation in cell culture medium (medium) and in PBS without ectoine (PBS) for 24 hours, HaCaT cells show a Claudin-1 expression being lower by a factor of 3.

TABLE 5
Claudin-1 expression in a HaCaT epithelial monolayer

| Claudin-1 | Medium | Ectoine 50 mM | Ectoine 100 mM |
|---|---|---|---|
| expression | [Claudin-1] +/− SD | | |
| | 0.07 ng/ml +/− 0.013 | −0.28 ng/ml +/− 0.002 | 0.22 ng/ml +/− 0.006 |

Thus, ectoine induces an enhanced expression of Claudin-1 after heat stress. It is to be expected that the enhanced expression of Claudin-1 correlates with an increase of TEER proving strengthening of the barrier function of HaCaT cells after drying stress.

The present results show that compatible solutes, such as ectoine and its derivatives, stabilise the cell membrane and thus strengthen the barrier function of the respective epithelial cells, in particular epithelial tissues. Thus, the present results show that ectoine inhibits penetration of biobased noxae (Table 1, such as toxins and allergens.

1-22. (canceled)

23. A method for treating or preventing a disorder associated with an epithelial tissue barrier malfunction associated with a bio-based noxa, the method comprising:
(a) providing a subject suffering from or at risk of developing a disorder associated with an epithelial tissue barrier malfunction characterized by damage to a tight junction in at least one cell layer of an epithelial tissue; and
(b) administering to the subject an effective amount of a composition comprising at least one compatible solute or a solute mixture comprising at least two compatible solutes,
wherein the at least one compatible solute and the at least two compatible solutes are selected from the group consisting of compounds of Formula I, compounds of Formula II, physiologically compatible salts thereof, physiologically compatible salts of stereoisomeric forms thereof, and combinations thereof, and further wherein Formulas I and II have the following structures: 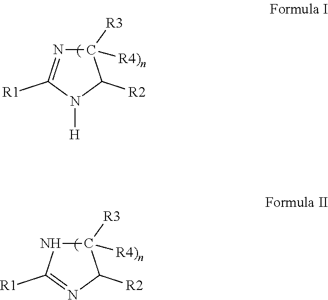
where:
R1 is H or alkyl;
R2 is H, COOH, COO-alkyl, or CO—NH—R5;
R3 is H or OH;
R4 is H or OH;
R5 is H, alkyl, an amino acid residue, a dipeptide residue, or a tripeptide residue;
n is 1, 2 or 3; and
alkyl is linear, cyclical, or branched C1-C4 alkyl.

24. The method of claim 23, wherein the epithelial tissue barrier malfunction comprises an impaired intercellular cell structure of at least one cell layer of outer epithelial tissue, transitional epithelial tissue, inner epithelial tissue, or any combination thereof.

25. The method of claim 23, wherein the epithelial tissue barrier malfunction comprises an impaired and reduced selective permeability barrier.

26. The method of claim 23, wherein the at least one bio-based noxa is selected from the group consisting of an animal noxa, a plant noxa, a noxa of an insect and/or a pest, a microbial noxa, an environmental noxa, and a food noxa, or any component and/or compound thereof.

27. The method of claim 26, wherein:
(a) the animal noxa comprises a noxa from a domestic animal, a cat, a dog, a pig, a rodent, a farm animal, a pig, a goat, an excretion therefrom, epithelia therefrom, and/or hair therefrom; and/or
(b) the plant noxa comprises a noxa from a flower pollen, a fruit organ, a sap, a plant secretion, a plant poison, a plant resin, a natural rubber, a latex, an odorous substance, a flavoring substance, a toxin, a stinging hair, a hook, a needle, a spine, and/or a component and/or compound therefrom; and/or
(c) the noxa of an insect and/or a pest comprises a noxa from a mite, a dust mite, an excretion of an insect, pest, or parasite; a bee glue; a bee venom; a wasp venom; a spider venom; a pest bite; a mosquito sting; a horsefly sting; an ant sting and/or bite; and/or a component and/or compound therefrom; and/or
(d) the microbial noxa comprises a noxa from a microorganism, a fungus, a yeast, a Malassezia species, a bacterium, a Staphylococcus aureus, a mold spore, a mold, a bacterial toxin, a delta toxin, an antibiotic, a virus, a mycotoxin, and/or a component and/or compound therefrom; and/or
(f) the environmental noxa is photodynamic exposure to light, wind, a seasonal temperature fluctuation, and an odor; and/or
(g) the food noxa comprises a noxa from a nut, a peanut, a hazelnut, a wine, a cow's milk, wheat, soy, a hen's egg, a protein, a fish, a shellfish, a crustacean, a mollusk, a raw vegetable, a raw fruit, and/or components and/or compounds therefrom.

28. The method of claim 23, wherein the bio-based noxa is peptide-based.

29. The method of claim 23, wherein the epithelial tissue comprises:
outer epithelial tissue selected from the group consisting of skin, outer skin, scalp, epidermis, nail bed, nail body, cornea and conjunctiva of the eye, outer ear, external auditory canal, and lips;
transitional epithelial tissue selected from the group consisting of oral cavity, oral mucosa, gingiva, tongue, tongue mucosa, upper respiratory tract, nasal cavity, paranasal sinuses, nasal mucosa, voice flaps, throat, and genitals; and/or
inner epithelial tissue selected from the group consisting of lower respiratory tract, trachea, bronchial tubes, bronchial tree, lungs, inner endothelial tissue, continuous endothelium, endothelium of heart blood vessels, heart lymph vessels, esophagus, gastric mucosa, and small/intestinal mucosa.

30. The method of claim 23, wherein the effective amount of the composition is 1 mM to 1 M of the at least one compatible solute.

31. The method of claim 23, wherein the epithelial tissue is characterized by a reduced transepithelial electric resistance (TEER), measured in ohms, as compared to non-damaged epithelial tissue of the same type.

32. The method of claim 23, wherein the epithelial tissue is characterized by an increased permeability for the bio-based noxa as compared to a non-damaged epithelial tissue of the same type.

33. The method of claim 23, wherein the epithelial tissue is characterized by reduced expression of at least one claudin protein family member as compared to a non-damaged epithelial tissue of the same type.

34. The method of claim 23, wherein the epithelial tissue barrier malfunction is associated with a symptom selected from the group consisting of itching; skin discoloration; calor; fever; redness; dry skin; ring-shaped skin alterations;
blisters; pustules, papules, and pimples, with or without suppuration; abscesses;
fistulas; rash; scabs; raisings; swellings; scratches; stings; sloughing;
desquamation; wheals; angioedema; Quincke edema; urticaria; plaques; ulcers;
furuncles; carbuncles; eczemas, and Baghdad boils.

35. The method of claim 23, wherein the epithelial tissue barrier malfunction is associated with parasitic, bacterial, and/or viral disease; mycoses; lesions;
dryness; irritation; inflammation; and/or hypersensitivity and/or allergic reaction of the outer epithelial tissue, the transitional epithelial tissue, and/or the inner epithelial tissue.

36. The method of claim 23, wherein the epithelial tissue barrier malfunction is associated with a disease of an outer epithelial tissue and/or a transitional epithelial tissue selected from the group consisting of:
outer epithelial tissue diseases selected from the group consisting of diseases of the skin and the subcutis, infections of the skin and/or of the subcutis, mycoses, dry skin, contact allergies, dermatitis, eczema, neurodermatitis, psoriasis, urticaria, herpes, orofacial herpes, diseases of the eye, conjunctivitis, keratitis, diseases of the outer ear, inflammation of the outer ear, inflammation of the outer auditory canal, Otitis externa, and physical impairment of and/or damage to the skin structure comprising injuries, stings, slashes, scratches, abrasions, burns and/or chemical burns, each triggered by at least one biobased noxa; and
transitional epithelial tissue diseases selected from the group consisting of allergic reaction of the nasal mucosa, allergic reactions of at least one mucosa of the oral cavity, dry nasal mucosa, diseases of the oral cavity, cysts, phlegmons, and/or abscesses of the oral mucosa, allergic lesions of the oral mucosa and/or of the tongue, diseases of the upper respiratory tract, nasal sinusitis, allergic rhinitis, allergic rhinopathy, tonsillitis, inflammations and infections of the mucosa of mouth, gingiva, tongue, throat, nose and/or genitals.

37. The method of claim 23, wherein the composition comprises at least one compatible solute selected from the group consisting of S-ectoine, R-ectoine, (S,S)-hydroxyectoine, (S,R)-hydroxyectoine, (R,S)-hydroxyectoine, (R,R)-hydroxyectoine and S-homoectoine, physiologically compatible salts of 5-ectoine, R-ectoine, (S,S)-hydroxyectoine, (S,R)-hydroxyectoine, (S,R)-hydroxyectoine, (R,S)-hydroxyectoine, (R,S)-hydroxyectoine, (R,R)-hydroxyectoine, and S-homoectoine, amides thereof, and esters thereof, or any combination thereof.

38. The method of claim 23, wherein the at least one compatible solute or the solute mixture comprising at least two compatible solutes is present in the composition in an amount of greater than or equal to 0.0001% by weight to less than or equal to 50% by weight, based on the total content of the composition.

39. The method of claim 23, wherein the composition is present as a medical product and/or a cosmetic, and/or as an additive thereto.

40. The method of claim 23, wherein the composition is present in a solid form, a liquid form, or as a mixture, wherein:
i) the solid form is selected from the group consisting of a powder, a lyophilisate, a tablet, a granule, a film-coated tablet, a dragee, a capsule, an effervescent tablet, a powder, and a soap;
ii) the liquid form is selected from the group consisting of a solution, an injection, an infusion, a tincture, an infusion solution, a suspension, an emulsion, an application, a foam, and a cream; and
iii) the mixture is selected from the group consisting of a spray, an aerosol, an ointment, a paste inhalant, and a capsule.
